(19) United States
(12) Patent Application Publication
(10) Pub. No.: US 2026/0137403 A1
(43) Pub. Date: May 21, 2026

(54) ASPIRATION CATHETER WITH ENHANCED CLOT RETENTION AND IMPROVED CATHETER CONSTRUCTION

(71) Applicant: Imperative Care, Inc., Campbell, CA (US)

(72) Inventors: Miranda M. Ray, San Jose, CA (US); Kevin Gabriel Kuehn, San Jose, CA (US); Liam Patrick Burns, Columbus, OH (US); Ivan Vega, Campbell, CA (US); Mathurin Boutte, San Jose, CA (US); Mark William McElligott, San Jose, CA (US)

(21) Appl. No.: 19/391,990

(22) Filed: Nov. 17, 2025

Related U.S. Application Data
(60) Provisional application No. 63/722,032, filed on Nov. 18, 2024.
(60) Provisional application No. 63/912,093, filed on Nov. 5, 2025.

(51) Int. Cl. A61B 17/22; A61B 17/00; A61M 25/00; A61M 25/01

(52) U.S. Cl. CPC A61B 17/22; A61M 25/0012; A61M 25/0045; A61M 25/005; A61M 25/007; A61M 25/0108; A61B 2017/00137; A61B 2017/00561; A61B 2017/00778; A61B 2017/22079; A61B 2217/005; A61M 2025/006; A61M 2202/0014; A61M 2202/06; A61M 2202/09; A61M 2205/32; A61M 2207/00; A61M 2210/12

(57) ABSTRACT

An aspiration catheter may include a tubular body having a proximal end, a distal end, and a lumen extending therethrough. The distal end of the tubular body can be configured to provide aspiration in a first aspiration mode to apply a tension force to a clot positioned near the distal end along a longitudinal axis of the tubular body. The aspiration catheter can be configured to provide aspiration in a second aspiration mode at least when the clot is positioned within the distal end of the tubular body to apply a second force to the clot along a second axis different from the longitudinal axis. The catheter can include a stent-like backbone positioned along a sidewall of the catheter. The stent-like backbone can include a metallic support having a continuous gap extending longitudinally along a length of the backbone.

### INCORPORATION BY REFERENCE TO ANY PRIORITY APPLICATIONS

Any and all applications for which a foreign or domestic priority claim is identified in the application data sheet as filed with the present application are hereby incorporated by reference under 37 C.F.R. §1.57. The present application claims priority to U.S. Provisional Patent Application No. 63/722032, filed Nov. 18, 2024, titled ASPIRATION CATHETER WITH ENHANCED CLOT RETENTION, and U.S. Provisional Patent Application No. 63/912093, filed Nov. 5, 2025, titled ASPIRATION CATHETER WITH ENHANCED CLOT RETENTION AND IMPROVED CATHETER CONSTRUCTION, the entire content of each of which is incorporated by reference herein for all purposes and forms as part of this specification.

### BACKGROUND

Stroke is the third most common cause of death in the United States and the most disabling neurologic disorder. Approximately 700,000 patients suffer from stroke annually. Stroke is a syndrome characterized by the acute onset of a neurological deficit that persists for at least 24 hours, reflecting focal involvement of the central nervous system, and is the result of a disturbance of the cerebral circulation. Its incidence increases with age. Risk factors for stroke include systolic or diastolic hypertension, hypercholesterolemia, cigarette smoking, heavy alcohol consumption, and oral contraceptive use.

Hemorrhagic stroke accounts for 20% of the annual stroke population. Hemorrhagic stroke often occurs due to a rupture of an aneurysm or arteriovenous malformation bleeding into the brain tissue, resulting in cerebral infarction. The remaining 80% of the stroke population are ischemic strokes and are caused by occluded vessels that deprive the brain of oxygen-carrying blood. Ischemic strokes are often caused by emboli or pieces of thrombotic tissue that have dislodged from other body sites or from the cerebral vessels themselves to occlude in the narrow cerebral arteries more distally. When a patient presents with neurological symptoms and signs which resolve completely within 1 hour, the term transient ischemic attack (TIA) is used. Etiologically, TIA and stroke share the same pathophysiologic mechanisms and thus represent a continuum based on persistence of symptoms and extent of ischemic insult.

Emboli occasionally form around the valves of the heart or in the left atrial appendage during periods of irregular heart rhythm and then are dislodged and follow the blood flow into the distal regions of the body. Those emboli can pass to the brain and cause an embolic stroke. As will be discussed below, many such occlusions occur in the middle cerebral artery (MCA), although such is not the only site where emboli come to rest.

When a patient presents with neurological deficit, a diagnostic hypothesis for the cause of stroke can be generated based on the patient's history, a review of stroke risk factors, and a neurologic examination. If an ischemic event is suspected, a clinician can tentatively assess whether the patient has a cardiogenic source of emboli, large artery extracranial or intracranial disease, small artery intraparenchymal disease, or a hematologic or other systemic disorder. A head CT scan is often performed to determine whether the patient has suffered an ischemic or hemorrhagic insult. Blood would be present on the CT scan in subarachnoid hemorrhage, intraparenchymal hematoma, or intraventricular hemorrhage.

Traditionally, emergent management of acute ischemic stroke consisted mainly of general supportive care, e.g. hydration, monitoring neurological status, blood pressure control, and/or anti-platelet or anti-coagulation therapy. In 1996, the Food and Drug Administration approved the use of Genentech Inc.'s thrombolytic drug, tissue plasminogen activator (t-PA) or Activase®, for treating acute stroke. A randomized, double-blind trial, the National Institute of Neurological Disorders and t-PA Stroke Study, revealed a statistically significant improvement in stoke scale scores at 24 hours in the group of patients receiving intravenous t-PA within 3 hours of the onset of an ischemic stroke. Since the approval of t-PA, an emergency room physician could, for the first time, offer a stroke patient an effective treatment besides supportive care.

However, treatment with systemic t-PA is associated with increased risk of intracerebral hemorrhage and other hemorrhagic complications. Patients treated with t-PA were more likely to sustain a symptomatic intracerebral hemorrhage during the first 36 hours of treatment. The frequency of symptomatic hemorrhage increases when t-PA is administered beyond 3 hours from the onset of a stroke. Besides the time constraint in using t-PA in acute ischemic stroke, other contraindications include the following: if the patient has had a previous stroke or serious head trauma in the preceding 3 months, if the patient has a systolic blood pressure above 185 mm Hg or diastolic blood pressure above 110 mmHg, if the patient requires aggressive treatment to reduce the blood pressure to the specified limits, if the patient is taking anticoagulants or has a propensity to hemorrhage, and/or if the patient has had a recent invasive surgical procedure. Therefore, only a small percentage of selected stroke patients are qualified to receive t-PA.

Obstructive emboli have also been mechanically removed from various sites in the vasculature for years. Mechanical therapies have involved capturing and removing the clot, dissolving the clot, disrupting and suctioning the clot, and/or creating a flow channel through the clot. One of the first mechanical devices developed for stroke treatment is the MERCI Retriever System (Concentric Medical, Redwood City, Calif.). A balloon-tipped guide catheter is used to access the internal carotid artery (ICA) from the femoral artery. A microcatheter is placed through the guide catheter and used to deliver the coil-tipped retriever across the clot and is then pulled back to deploy the retriever around the clot. The microcatheter and retriever are then pulled back, with the goal of pulling the clot, into the balloon guide catheter while the balloon is inflated and a syringe is connected to the balloon guide catheter to aspirate the guide catheter during clot retrieval. This device has had initially positive results as compared to thrombolytic therapy alone.

Other thrombectomy devices utilize expandable cages, baskets, or snares to capture and retrieve clot. Temporary stents, sometimes referred to as stentrievers or revascularization devices, are utilized to remove or retrieve clot as well as restore flow to the vessel. A series of devices using active laser or ultrasound energy to break up the clot have also been utilized. Other active energy devices have been used in conjunction with intra-arterial thrombolytic infusion to accelerate the dissolution of the thrombus. Many of these devices are used in conjunction with aspiration to aid in the removal of the clot and reduce the risk of emboli. Suctioning of the clot has also been used with single-lumen catheters and syringes or aspiration pumps, with or without adjunct disruption of the clot. Devices which apply powered fluid vortices in combination with suction have been utilized to improve the efficacy of this method of thrombectomy. Finally, balloons or stents have been used to create a patent lumen through the clot when clot removal or dissolution was not possible.

Notwithstanding the foregoing, there remains a need for new devices and methods for treating vasculature occlusions in the body, including acute ischemic stroke and occlusive cerebrovascular disease. In particular, as will be discussed in more detail below, there is a need for enhanced aspiration catheters for use in vascular procedures.

### SUMMARY

There is provided in accordance with one aspect of the present disclosure an aspiration catheter. The aspiration catheter can include a tubular body including a proximal end, a distal end, and a lumen extending therethrough, wherein the distal end of the tubular body is configured to provide aspiration in a first aspiration mode to apply a tension force to a clot positioned near the distal end along a longitudinal axis of the tubular body; wherein the aspiration catheter is configured to provide aspiration in a second aspiration mode at least when the clot is positioned within the distal end of the tubular body to apply a second force to the clot along a second axis different from the longitudinal axis.

In some aspects, the second axis can be transverse to the longitudinal axis. The second force applied to the clot along the second axis can be from within the lumen. In some cases, the lumen can provide a first, axial flow path for applying the tension force to the clot, and a structure within the lumen can define a second flow path for applying a radially outwardly directed flow path. In some aspects, the structure includes a barrier within the lumen and spaced radially inwardly apart from an inner surface of the tubular body. The barrier can be porous. In some cases, the aspiration catheter can include a plurality of holes on an inner surface within the lumen of the tubular body, wherein the plurality of holes are configured to provide aspiration in the second aspiration mode to apply the second force to the clot. In some aspects, the aspiration catheter can include an annular band positioned at or near the distal end of the tubular body, the annular band including the plurality of holes. The first aspiration mode can be configured to be active when at least a portion of an opening at the distal end of the tubular body is unobstructed by the clot. In some cases, the second aspiration mode can be configured to be active when the clot is corked to the opening at the distal end of the tubular body. In some aspects, aspiration provided by the aspiration catheter can be configured to transition from the first aspiration mode to the second aspiration mode when the clot gets corked to the opening at the distal end of the tubular body. Aspiration provided by the aspiration catheter can be configured to transition from the second aspiration mode to the first aspiration mode when the clot gets uncorked from the opening at the distal end of the tubular body. In some cases, the aspiration catheter can include a solid surface on an inner surface of the tubular body, wherein the solid surface forms a leak path configured to apply aspiration.

There is also provided in accordance with one aspect of the present disclosure, a method of aspirating a clot. The method can include positioning a distal end of an aspiration catheter adjacent a clot; applying an aspiration force to the clot along a first axis to draw the clot at least partially into the catheter; and applying a retention force to the clot along a second axis to enhance retention of the clot to the catheter.

In some aspects, the retention force can be applied along the second axis in response to corking of a clot in the catheter.

There is also provided in accordance with one aspect of the present disclosure, a catheter configured to apply a first, aspiration force and a second, retention force to a clot, including: an elongate, flexible tubular body having a first flow path for applying an axial aspiration force to a clot; and a second flow path in the tubular body, for applying a radially outwardly directed retention force to the clot.

In some aspects, the second flow path can be spaced apart from the first flow path by a porous barrier. The barrier can include a mesh. In some cases, the barrier can include a porous wall. In some aspects, the second flow path can be defined between the clot and an inside surface of the flexible tubular body. The inside surface of the flexible tubular body can include a plurality of spacers to space the clot apart from the inside surface. In some cases, the plurality of spacers can include a plurality of axially extending grooves separated by a plurality of axially extending ridges.

There is also provided in accordance with one aspect of the present disclosure, an aspiration catheter including: a tubular body including a proximal end, a distal end, and a lumen extending therethrough; and an annular band positioned at or near the distal end of the tubular body, wherein the annular band includes: an outer layer, an inner layer including a plurality of holes, and a gap between the outer layer and the inner layer; wherein the gap is in fluid communication with the lumen via the plurality of holes; wherein upon application of aspiration in the lumen, a first subset of the plurality of holes is configured to engage a clot received at the distal end of the tubular body and a second subset of the plurality of holes at least partially unobstructed by the clot is configured to remain in fluid communication with the lumen.

In some aspects, the distal end of the gap can be in fluid communication with the lumen. A distal face of the annular band and a longitudinal axis of the tubular body can form an angle from about 30° to about 80°. In some cases, a proximal face of the annular band and a longitudinal axis of the tubular body can form an angle of about 90°. In some aspects, the plurality of holes can include a plurality of chevron-shaped holes. The plurality of holes can include a plurality of circular-shaped holes. In some cases, the plurality of holes can include a plurality of circular-shaped holes. In some aspects, the annular band can be configured such that, when the clot is not corked to the distal end of the tubular body, aspiration occurs only at the distal end of the tubular body and not through the plurality of holes of the annular band.

There is also provided in accordance with one aspect of the present disclosure, an aspiration catheter including: a tubular body including a proximal end, a distal end, and a lumen extending therethrough; an inner wall surface at or near the distal end of the tubular body including a plurality of holes, and a gap between the inner wall surface and an outer surface of the tubular body; wherein the gap is in fluid communication with the lumen via the plurality of holes; wherein upon application of aspiration in the lumen, at least some of the plurality of holes are configured to apply aspiration radially to a clot received at the distal end of the tubular body.

In some aspects, the plurality of holes can extend longitudinally and circumferentially in a repeating pattern along a distal portion of the tubular body. The plurality of holes can be provided in an annular band positioned near the distal end of the tubular body. In some cases, the annular band can be radiopaque. In some aspects, the plurality of holes can be provided in a coining insert positioned near the distal end of the tubular body. The gap can be positioned between the tubular body and the coining insert.

There is also provided in accordance with one aspect of the present disclosure, a method of removing a clot from the vasculature of a patient, the method including: advancing an aspiration catheter through the vasculature of the patient until a distal end of the aspiration catheter is adjacent a location of the clot; applying aspiration via a lumen of the catheter to capture at least a portion of the clot within the lumen; and while the clot is corked to the distal end of the aspiration catheter, applying aspiration through a plurality of holes on an inner surface within the lumen of the aspiration catheter to apply a radial aspiration force to the clot.

In some aspects, while the clot is corked to the distal end of the aspiration catheter, aspiration can be applied through a first subset of holes and a second subset of holes fluidly connected by a gap positioned on a distal portion of the aspiration catheter; wherein the first subset of holes is obstructed by the clot and is configured to maintain the clot engaged to the first subset of holes; and wherein the second subset of holes is at least partially unobstructed by the clot and is configured to stay in fluid communication with the lumen. The plurality of holes can be provided on an inner surface of an annular band positioned in a distal portion of the aspiration catheter. In some cases, advancing the aspiration catheter through the vasculature of the patient until a distal end of the aspiration catheter is adjacent the location of the clot can include advancing the aspiration catheter inside a second catheter and beyond a distal end of the second catheter. In some aspects, the method can include, with the clot secured to the distal end of the aspiration catheter, retracting the aspiration catheter through the second catheter at least until the distal end of the aspiration is positioned inside the second catheter. The method can include removing the aspiration catheter and the second catheter from the vasculature of the patient. In some cases, when the clot is not corked to the distal end of the aspiration catheter, aspiration can be applied only at the distal end of the catheter and not through the plurality of holes.

There is also provided in accordance with one aspect of the present disclosure, a backbone for a catheter. The backbone can include a metallic support configured to be positioned over an inner liner, the metallic support including a continuous gap extending longitudinally along a length of the metallic support; wherein a transverse cross-section of the metallic support defines a c-shape.

In some aspects, the continuous gap is not linear.

The metallic support can include a plurality of open rings, each of the plurality of open rings including: a first band; a second band; at least one beam connecting the ends of the first band and the second band; and at least one connection connecting the first band and the second band.

In some cases, each open ring of the plurality of open rings can be attached to another open ring by at least one strut.

In some aspects, each open ring of the plurality of rings can include a pair of beams, and wherein the pair of beams is separated by a gap.

The gap of each pair of beams can be offset from about 15° to about from 55° from a preceding gap or a subsequent gap.

In some cases, the gap of each pair of beams can be offset by about 35°from a preceding gap or a subsequent gap.

In some aspects, the at least one beam can include a narrow-arc shape.

The at least one beam can include a wide-arc shape.

In some cases, every other beam can include a narrow-arc shape and every other beam can include a wide-arc shape.

In some aspects, the metallic support can include a first portion and a second portion separate from the first portion, each of the first portion and the second portion including: a plurality of ringlets, each of the plurality of ringlets including: a first band; a second band; and at least one beam connecting the ends of the first band and the second band.

Each ringlet of the plurality of ringlets can be attached to another ringlet by at least one strut.

In some cases, each ringlet of the of the plurality of ringlets can include a pair of beams.

In some aspects, each beam of the pair of beams of the first portion can be separated from each beam of the pair of beams of the second portion by a gap.

The at least one beam can include a narrow-arc shape.

In some cases, the at least one beam can include a wide-arc shape.

In some aspects, every other beam can include a narrow-arc shape and every other beam can include a wide-arc shape.

In some aspects, the techniques described herein relate to a catheter including a stent-like backbone, the stent-like backbone including: a plurality of open rings, each of the plurality of open rings including: a first band; a second band; at least one beam connecting the ends of the first band and the second band; and at least one connection connecting the first band and the second band.

In some aspects, the techniques described herein relate to a catheter, wherein each open ring of the plurality of open rings is attached to another open ring by at least one strut.

In some aspects, the techniques described herein relate to a catheter, wherein each open ring of the plurality of rings includes a pair of beams, and wherein the pair of beams is separated by a gap.

In some aspects, the techniques described herein relate to a catheter, wherein the gap of each pair of beams is offset from about 350 to about from 550 from a preceding gap or a subsequent gap.

In some aspects, the techniques described herein relate to a catheter, wherein the gap of each pair of beams is offset by about 350 from a preceding gap or a subsequent gap.

In some aspects, the techniques described herein relate to a catheter, wherein the at least one beam includes a narrow-arc shape.

In some aspects, the techniques described herein relate to a catheter, wherein the at least one beam includes a wide-arc shape.

In some aspects, the techniques described herein relate to a catheter, wherein every other beam includes a narrow-arc shape and every other beam includes a wide-arc shape.

There is also provided in accordance with one aspect of the present disclosure, a catheter including a stent-like backbone. The stent-like backbone can include a first portion and a second portion separate from the first portion, each of the first portion and the second portion including: a plurality of ringlets including: a first band; a second band; and at least one beam connecting the ends of the first band and the second band.

In some aspects, each ringlet of the plurality of ringlets can be attached to another ringlet by at least one strut.

Each ringlet of the of the plurality of ringlets can include a pair of beams.

In some cases, each beam of the pair of beams of the first portion can be separated from each beam of the pair of beams of the second portion by a gap.

In some aspects, the at least one beam can include a narrow-arc shape.

The at least one beam can include a wide-arc shape.

In some cases, every other beam can include a narrow-arc shape and every other beam can include a wide-arc shape.

There is also provided in accordance with one aspect of the present disclosure, a method of manufacturing a catheter. The method can include providing an inner liner; with a backbone in an open position, loading the backbone onto the inner liner; and closing the backbone thereby securing the backbone to the inner liner; wherein the backbone includes a metallic support including at least one continuous gap extending longitudinally along a length of the backbone.

In some aspects, the at least one continuous gap is not linear.

The method can also include positioning a jacket over the inner liner and the backbone, wherein the jacket includes a plurality of polymer segments.

There is also provided in accordance with one aspect of the present disclosure, a backbone for a catheter. The backbone can include an elongate metallic support configured to be positioned over an inner liner of the catheter, the elongate metallic support including, a plurality of open rings arranged along the metallic support, each open ring including a first band, a second band, and a pair of beams connecting ends of the first band and the second band, wherein the pair of beams are separated by a gap; a plurality of struts including at least one strut connecting each open ring to an adjacent open ring; and a plurality of connections including at least one connection within each open ring connecting the first band to the second band; wherein the at least one strut is oriented at a non-parallel angle relative to a longitudinal axis of the backbone; and wherein the at least one connection is oriented at a non-parallel angle relative to the longitudinal axis.

In some aspects, an angle of successive struts of the plurality of struts relative to the longitudinal axis can decrease from a distal portion of the backbone toward a proximal portion of the backbone.

The at least one strut can be positioned on a distal portion of the backbone, and the non-parallel angle of the at least one strut relative to the longitudinal axis of the backbone can be between about 50° and about 60°.

In some cases, the elongate metallic support can include at least one additional strut positioned on a proximal portion of the backbone, wherein an angle or the at least one additional strut relative to the longitudinal axis of the backbone is between about 30° and about 40°.

In some aspects, each open ring can be connected to at least another open ring by the first strut and a second strut, and wherein the first strut and the second strut are oriented in opposite directions relative to each other.

The first strut and the second strut can be positioned between about 150° to about 180° from each other around a circumference of the backbone.

In some cases, a width of each of the first band and the second band can be larger than a width of each beam of the pair of beams.

In some aspects, a width of each of the first band and the second band is between about 1.5 to about 3 times a width of each beam of the pair of beams.

The backbone can include a pattern in which a first pair of consecutive struts of the plurality of struts on a first side of the backbone are oriented in a first direction, and an adjacent second pair of consecutive struts of the plurality of struts on the first side are oriented in a second direction different from the first direction.

In some cases, the backbone includes a pattern in which a first pair of consecutive connections of the plurality of connections are oriented in a first direction, and an adjacent second pair of consecutive connections of the plurality of connections are oriented in a second direction different from the first direction.

In some aspects, the gap between each pair of beams can be offset from about 35° to about from 55° from a preceding gap or a subsequent gap.

Spacing between adjacent open rings can decrease in a proximal direction such that a first spacing between adjacent open rings on a distal portion of the backbone is greater than a second spacing between adjacent open rings on a proximal portion of the backbone.

In some cases, the backbone can include an annular band secured to a distal end of the elongate metallic support.

In some aspects, the annular band can include a plurality of slots.

There is also provided in accordance with one aspect of the present disclosure, a catheter including: a tubular body defining a central lumen and including, an inner liner, a stent-like backbone positioned over the inner liner, an outer layer positioned over the inner liner and the stent-like backbone, an annular band secured to a distal end of the stent-like backbone and including a plurality of slots, a spacer including a plurality of cavities and positioned over the annular band, and a marker band positioned over the annular band and the spacer; wherein the plurality of cavities of the spacer are in fluid communication with the central lumen via the plurality of slots; and wherein upon application of aspiration in the central lumen, a first subset of the plurality of slots is configured to engage a clot received at the distal end of the tubular body and a second subset of the plurality of slots at least partially unobstructed by the clot is configured to remain in fluid communication with the central lumen.

In some aspects, a distal end of the inner liner can be positioned proximal to a proximal end of the annular band.

The stent-like backbone can include an elongate metallic support including: a continuous gap extending longitudinally along a length of the metallic support; a plurality of open rings arranged along the metallic support, each open ring including a first band, a second band, and a pair of beams connecting ends of the first band and the second band, wherein the pair of beams are separated by a gap; at least one strut connecting each open ring to an adjacent open ring; and at least one connection within each open ring connecting the first band to the second band.

In some cases, the at least one strut can be oriented at a non-parallel angle relative to a longitudinal axis of the backbone; and wherein the at least one connection is oriented at a non-parallel angle relative to the longitudinal axis.

In some aspects, the outer layer can include one or more polymer segments.

The spacer can include a proximal rim, a distal rim, and one or more support structures defining the plurality of cavities, wherein the proximal rim and the distal rim are connected to each other by the one or more support structures.

In some cases, the one or more support structures can include one or more longitudinal dams extending parallel to a longitudinal axis of the backbone, and one or more curved support structures.

In some aspects, the one or more curved support structures can allow cross communication among the plurality of cavities via the plurality of slots.

A thickness of the spacer can be between about 0.001 in to about 0.02 in.

### BRIEF DESCRIPTION OF THE DRAWINGS

FIGS. 1A-1E show an example of an aspiration catheter with a porous tip.

FIG. 2 shows an example of a porous tip for an aspiration catheter.

FIGS. 3A-3B show examples of porous tips for an aspiration catheter.

FIG. 4 shows an example of an inner layer for a porous tip.

FGS. 5A-5D show an example of an aspiration catheter with a porous tip in the process of engaging a blood clot.

FIG. 6A shows an example of a coining insert for a porous tip.

FIG. 6B shows the coining tip of FIG. 6A secured to a porous band.

FIG. 7 shows an example of an annular band for an aspiration catheter having a funnel shape.

FIGS. 8A and 8B show an example of an aspiration catheter with a molded tip.

FIG. 9 shows an example of a catheter having an inner liner with a plurality of layers.

FIGS. 10A and 10B show an example of a catheter with a backbone.

FIGS. 10C-10E show an example of a backbone for a catheter.

FIGS. 11A and 11B show an example of a backbone for a catheter.

FIGS. 12A and 12B show an example of a backbone for a catheter.

FIGS. 13A-13D show an example of a backbone for a catheter.

FIG. 13E shows catheter with a backbone.

FIGS. 14A-14B show an example of a backbone for a catheter.

### DETAILED DESCRIPTION

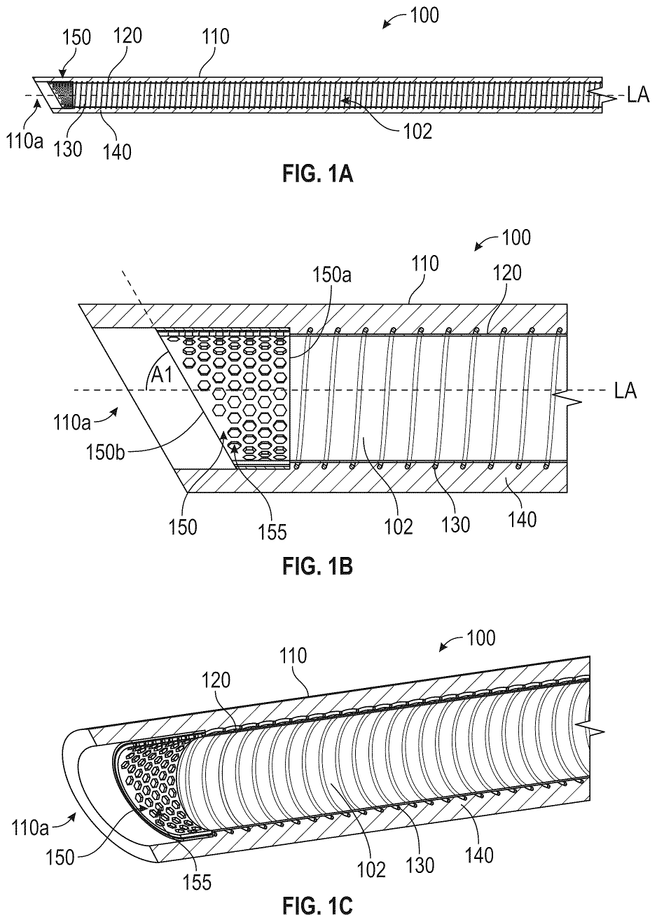

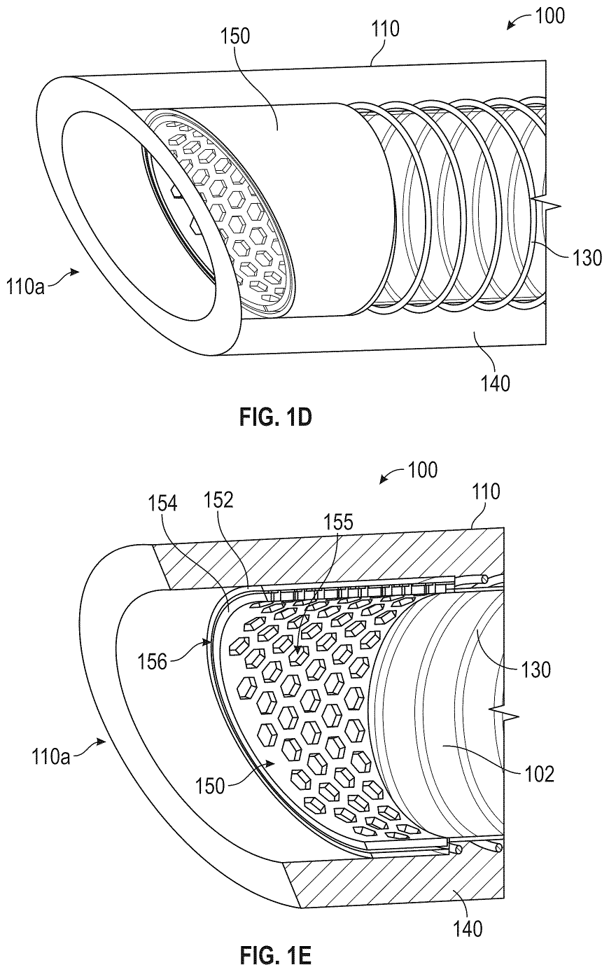

FIGS. 1A-1E illustrate an example of a catheter in accordance with one aspect of the present disclosure. The catheter 100 can include an inner liner, a reinforcement element, and/or an outer jacket. For example, the catheter 100 can have a tubular body 110 including a distal end 110a, an inner liner 120, a reinforcing element 130, an outer jacket 140, and/or an annular band (also referred herein to as a porous band) 150. In some cases, the reinforcing element 130 can include a braid and/or a coil. The reinforcing element 130 can be positioned radially outward of the inner liner 120. In some cases, the reinforcing element 130 can be embedded in the outer jacket 140, which can be positioned radially outward of the inner liner 120. The inner liner 120 and/or the reinforcing element 130 can terminate proximal to the annular band 150.

The annular band 150 can be radiopaque, which can beneficially allow visualization of the catheter 100 and/or the annular band 150 under fluoroscopy. In some cases, the annular band 150 can comprise a metallic material. For example, the annular band 150 can include a microfoam metallic material.

The catheter 100 can include a lumen 102 extending radially inward from the inner liner 120. The lumen 102 can extend an entire length of the insert catheter 100. The lumen 102 can extend from a proximal end of the tubular body 110 to the distal end 110a. The lumen 102 can receive other interventional devices, such as a guidewire and/or additional catheters. In some cases, the catheter 100 can be positioned inside and/or advanced through another catheter. In such cases, the catheter 100 may extend beyond a distal end of the catheter where the catheter 100 is positioned.

The catheter 100 can be in communication with a source of aspiration (e.g., vacuum). This can beneficially allow the catheter 100 to provide aspiration through the lumen 102. During an interventional procedure, aspiration can be applied to capture and/or remove clots from the vasculature of a patient. For example, aspiration can be applied to suction a clot into the lumen 102 of the catheter 100. As further described below, aspiration can be applied to allow the annular band 150 or other structure to assist in capturing a clot. This can beneficially prevent disengagement of clots from the catheter 100 and ensure safe removal of the clots from a patient.

In some cases incorporating an annular band, the annular band 150 can include an outer layer 152, an inner layer 154, and a gap 156 between the outer layer 152 and the inner layer 154, as illustrated in FIG. 1E. In some cases, the inner layer 154 can be porous. For example, the inner layer 154 can include a plurality of holes 155. The plurality of holes 155 can extend through an entire thickness of the inner layer 154. The lumen 102 and the gap 156 can be in fluid communication with each other via the plurality of holes 155 of the inner layer 154. In some cases, the plurality of holes 155 can be formed by laser cutting the inner layer 154. The plurality of holes 155 can include various shapes. For example, as shown in FIGS. 1A-1E, the plurality of holes 155 can include an hexagonal shape. As further described herein, the plurality of holes 155 can include a circular shape, a chevron shape, and/or any other shape. The annular band 150 can be positioned on a distal portion of the catheter 100. In some cases, the annular band 150 can be embedded within the tubular body 110.

In may be beneficial to minimize the material content of the annular band 150 as much as possible. Thus, in some cases, when manufacturing the annular band 150, the amount of material used in the annular band 150 can be reduced as much as possible while still allowing for the plurality of holes 155 to be formed in the annular band 150.

In some cases, and as illustrated in FIG. 1B, the annular band can include a proximal face 150a and a distal face 150b. The proximal face 150a can be positioned on a plane perpendicular to a longitudinal axis LA of the catheter 100. The distal face 150b can be positioned on a plane forming an angle A1 with the longitudinal axis LA of the catheter 100. In some cases, the angle A1 can be from about 20° to about 80°. For example, the angle A1 can be from about 30° to about 80°, from about 40° to about 70°, and/or from about 50° to about 60°.

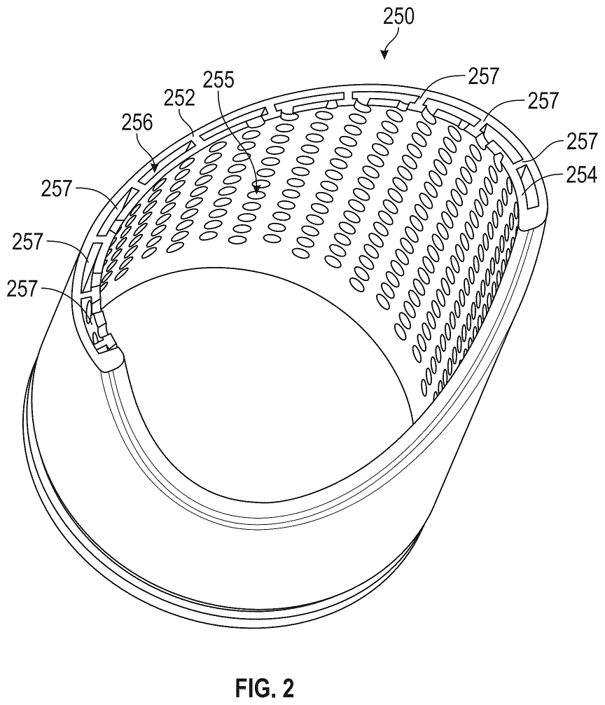

FIG. 2 illustrates a cross section of an example of an annular band 250. The annular band 250 can include an outer layer 252, an inner layer 254, and a gap 256 between the outer layer 252 and the inner layer 254. In some cases, the inner layer 254 can be porous. For example, the inner layer 254 can include a plurality of holes 255. The plurality of holes 255 can extend through an entire thickness of the inner layer 254. Any of the annular bands described herein can include a single-body construction. For instance, the annular band 250 can be 3D printed as one piece. This can beneficially reduce the risk of small components falling apart and/or ensure that the gap 256 between the outer layer 252 and the inner layer 254 is maintained during manufacturing and/or during use.

In some cases, the outer layer 252 and the inner layer 254 can be connected to each other via a plurality of walls 257. As shown in FIG. 2, the plurality of walls 257 can be positioned within the gap 256. The plurality of walls 257 can define a plurality of channels 258 within the gap 256. Each of the plurality of channels 258 can be in fluid communication with at least some of the plurality of holes 255. In some cases, each of the plurality of channels 258 may be separated from each other by the plurality of walls 257. The plurality of channels 258 can be arranged parallel to a longitudinal axis of the annular band. The plurality of holes 255 can be arranged in parallel rows that run parallel to the longitudinal axis of the annular band 250. In some cases, the plurality of channels 258 and/or the plurality of walls 257 can extend perpendicular to the longitudinal axis of the annular band 250.

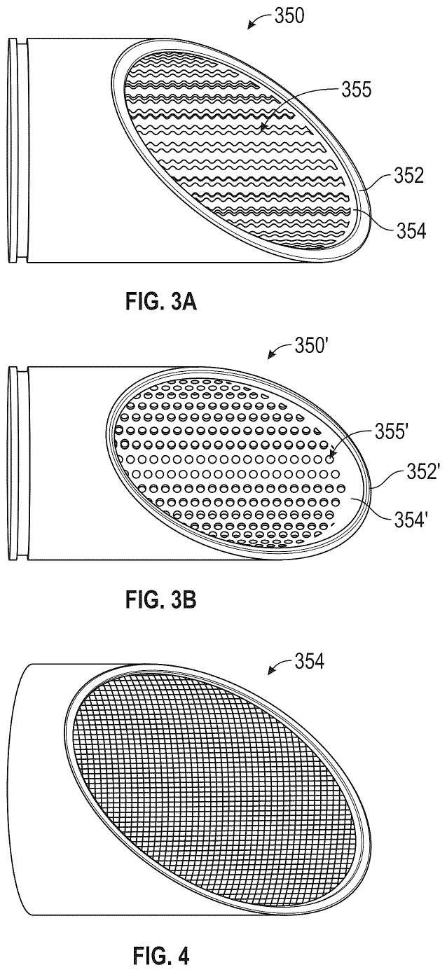

FIGS. 3A and 3B show examples of annular bands for an aspiration catheter. The annular band 350, which is shown in FIG. 3A, can include an outer layer 352, and inner layer 354, and a plurality of holes 355 along the inner layer 354. Unlike other plurality of holes described herein, the plurality of holes 355 can include a chevron shape, such as a repeating chevron shape. The plurality of holes 355 can extend along the inner layer 354 of the annular band 350. Each chevron shaped hole of the plurality of holes 355 can extend from a proximal end 350a of the annular band to a distal end 350b of the annular band 350 to form a number of parallel, repeating chevron shaped holes.

The annular band 350′, which is shown in FIG. 3B, can include an outer layer 352′, an inner layer 354′, and/or a plurality of holes 355′. The plurality of holes 355 can include a circular shape. The plurality of holes 355′ can extend along the inner layer 354′ of the annular band 350′. The plurality of holes 355′ can extend from a proximal end 350a′ of the annular band to a distal end 350b′ of the annular band 350.

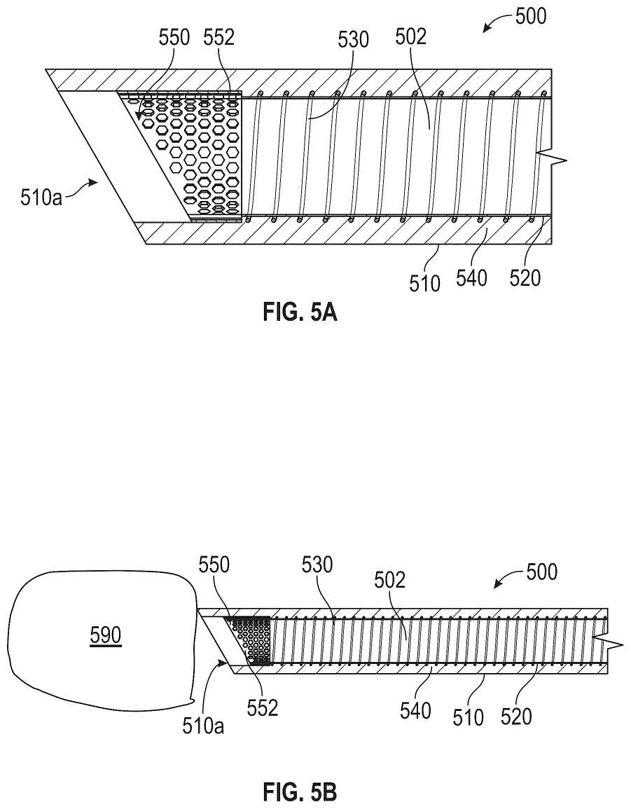

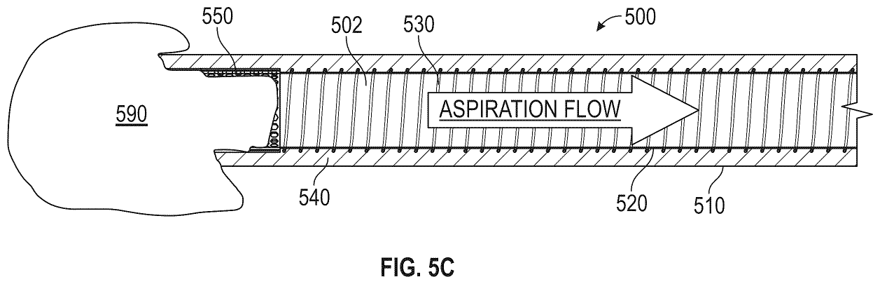

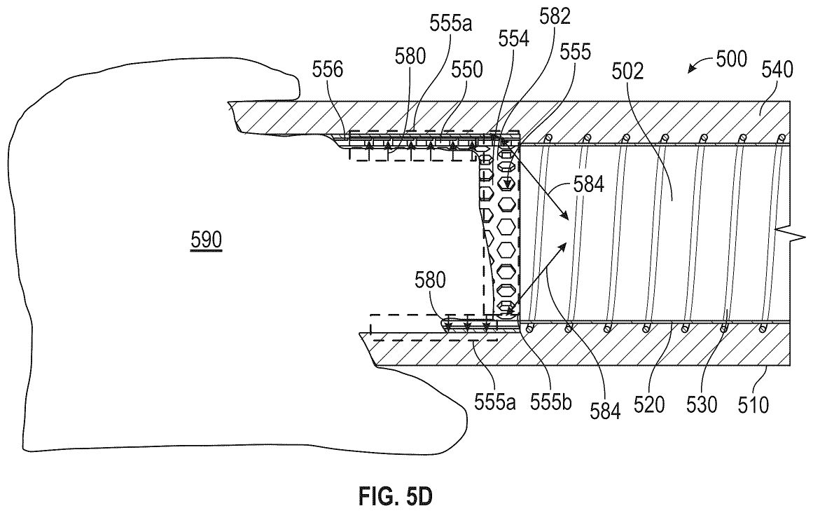

Any of the annular bands described herein can include a solid surface. In such cases, the annular bands may comprise a single layer with an outer surface and an interior surface. For instance, as shown in FIG. 4, an annular band 450 can include an interior surface with a solid surface 451. The solid surface 451 can include plurality of ridges, grooves, projections, standoffs, channels, and/or a combination thereof. The plurality of ridges, grooves, projections, standoffs, and/or channels can create a space between an interior wall of the catheter and a clot. In some cases, the solid surface 451 of the interior surface can help secure a clot and/or create leak paths for aspiration to be applied along the interior surface. Thus, the annular bands may not have separate layers (e.g., an inner layer and an outer layer). However, annular bands having two or more layers may include a solid surface as described in relation to FIG. 4 along at least the inner layer and/or the outer layer. In other cases, the solid surface as described herein may be applied to any inner surface of the tubular body, and need not be provided on an annular band.

Any of the catheters described herein can be used to capture and/or remove clots from the vasculature of a patient. For example, as shown in FIGS. 5A-5D, a catheter 500 with a annular band 550 can be used to capture a clot 590 within a vessel of a patient. The catheter 500 can be similar and/or identical to the catheter 100, which is described in relation to FIGS. 1A-1E. For instance, the catheter 500 can have a tubular body 510 including a distal end 510a, an inner liner 520, a reinforcing element 530, an outer jacket 540, a annular band 550, and/or a lumen 502. The annular band 550 can include an outer layer 552, an inner layer 554, and a gap 556 between the outer layer 552 and the inner layer 554. The inner layer 554 can include a plurality of holes 555.

During an interventional procedure, the catheter 500 can be inserted into the vasculature of a patient and advanced therethrough to position the distal end 510a of the tubular body 510 adjacent to a clot 590, as shown in FIG. 5B. Upon positioning of the distal end 510a of the tubular body 510 adjacent to the clot 590, aspiration can be activated to supply aspiration via the lumen 502.

As further described below, aspiration may be applied in more than one mode. The one or more aspiration modes may result in aspiration being applied in different directions and/or along different force vectors. For example, in a first aspiration mode, aspiration can expose the clot to a tension force (e.g., wherein aspiration is at least substantially parallel to the longitudinal axis of the catheter 500). The first aspiration mode may occur when the catheter 500 is initially positioned adjacent to the clot 590 and/or when the opening along the distal end 510a of the tubular body 510 is at least partially unobstructed by the clot 590. The first aspiration mode may allow for a quick and convenient initial engagement of the clot 590. In a second aspiration mode, aspiration can expose the clot to a second force (e.g., wherein aspiration is at least substantially perpendicular to the longitudinal axis of the catheter 500 and/or wherein the aspiration is along a second axis that can be transverse to the longitudinal axis of the catheter 500). This second force may be a retention force to enhance retention of the clot to the catheter. The retention force may be radially outwardly directed. As further described below, aspiration in the second aspiration mode may occur through the plurality of holes 555. The second aspiration mode may occur when the clot 590 is corked to the catheter 500. Thus, the catheter 500 may transition from the first aspiration mode to the second aspiration mode when the clot 590 is corked to the catheter 500. The catheter 500 may transition from the second aspiration mode to the first aspiration mode when the clot 590 is uncorked from the catheter 500. The second aspiration mode can allow the catheter 500 to maintain the clot 590 attached (e.g., secured) inside the lumen 502.

The aspiration provided by the lumen 502 can aspirate at least a portion of the clot 590 into the lumen 502, as shown in FIGS. 5C and 5D. The plurality of holes 555 along the inner layer 554 of the annular band 550 can provide aspiration within the tubular body 510. For instance, when a clot 590 is clogged to the distal end 510a of the tubular body 510, aspiration provided by the lumen 502 can allow at least some of the plurality of holes 555 to pull the clot 590 against the inner layer 554, as shown in FIG. 5D, thus securing the clot 590 to the catheter 500. This can beneficially prevent disengagement of the clot 590 from the catheter 500.

As shown in FIG. 5D, when a clot 590 is corked to the distal end 510a of the tubular body 510, aspiration can flow from a first subset of the plurality of holes 555 to a second subset of the plurality of holes 555 via the gap 556. For example, aspiration can flow from a first subset of holes 555a (which can be in contact with and/or at least partially obstructed by the clot 590) into the gap 556, as represented by arrows 580. This can cause the first subset of holes 555a to pull the clot 590 toward the inner layer 554. Further, aspiration can flow from the gap 556 to a second subset of holes 555b (which may not be in contact with and/or at least partially unobstructed by the clot 590), as represented by arrow 582. Aspiration can flow from the second subset of holes 555b to the lumen 502, as represented by arrows 584. Thus, aspiration flow within the catheter can flow both longitudinally along the lumen as well as radially outwardly.

The aspiration provided by the plurality of holes 555 can also dehydrate the clot 590 thus reducing the size of the clot 590. This can beneficially assist suction of the clot 590 into the lumen 502. The additional aspiration provided by the annular band 550 can also allow for the use of smaller catheters without sacrificing aspiration power and/or efficiency.

In cases where the catheter 500 is positioned inside and/or extended through another catheter, the aspiration provided by the plurality of holes 554a can prevent disengagement of the clot 590 from the catheter 500 when, for example, the catheter 500 is being retracted back into the other catheter and/or the catheter 500 is being removed from a patient.

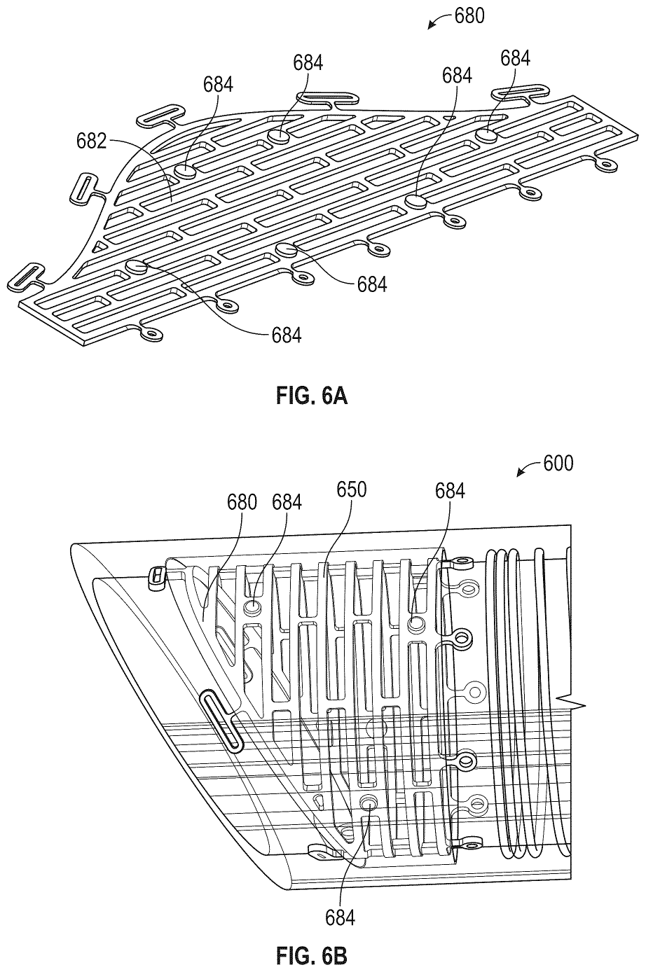

Any of the catheters described herein can include a coining insert. For example, as shown in FIG. 6A, a coining insert 680 can include a plurality of holes 682 and/or spacers 684 (also referred herein to as standoffs). The plurality of spacers 684 can separate the coining insert 680 from the interior wall of the catheter 600 and/or the interior surface of the annular band 650. The coining insert can be radiopaque. As shown in FIG. 6A, the coining insert can include a stent structure. The stent structure can be cut and/or etched to form one or more feet. The one or more feet can separate the coining insert from the interior wall of the catheter 600. In some cases, the stent structure can include one or more layers having different thicknesses. An etching masking method can be used to form the multi-thickness pattern.

In some cases, the coining insert 680 can be radially inward from an outer layer of an annular band 650 and/or embedded within a catheter 600, as shown in FIG. 6B. The coining insert 680 and an outer layer of the annular band 650 can be separated by a gap. The gap can be similar or identical to any of the gaps described hereon. In some cases, the plurality of holes 682 of the coining insert 680 can place a lumen of the catheter 600 and the gap in fluid communication with each other.

In some cases, catheters including a coining insert 680 may not include an annular band. In such cases, and as an example, the coining insert 680 can be positioned at or near the distal end of the catheter 600 (and/or a tubular body thereof). The plurality of spacers 684 can extend away from the coining insert 680 to provide a gap between an interior surface of the catheter 600 and the coining insert 680 when the coining insert 680 is positioned inside the catheter 600. The plurality of holes 682 of the coining insert 680 can place the gap and the lumen of the catheter 600 in fluid communication with each other. In some cases, the gap and/or grove between the coining insert 680 and an interior surface of the catheter 600 can be formed on the catheter 600.

Any of the annular bands described herein can be made of and/or include different materials. For example, in some cases, the annular bands can include an ePTFE porous membrane; a stent body (e.g., metallic: Steel, Nitinol, Tantalum; and/or non-metallic: Polyimide, PEEK, Nylon; high temperature polymers); a mesh braided metal extended from the tubular body 110, a nylon mesh fabric, a PTFE mesh fabric; a 3D printed metallic structure (e.g., steel), or a combination thereof. In some cases, the annular bands and/or the coining inserts may be manufactured using an electrical discharge machining (EDM) process.

Any of the annular bands described herein can undergo a laser drilling (or laser etching) process. For instance, laser drilling can be used to create a rough surface on an inner surface of the annular band. In some cases, the laser drilling process can include using a focused laser beam to lathe and/or ablate a tube to create one or more grooves, blind grooves, holes, and/or blind holes along the inner surface of the annular band. For example, one or more grooves and/or blind grooves can be formed along an inner surface of the of the annular band using a laser lathe. Additionally, or alternatively, one or more holes and/or blind holes can be formed along the inner surface of the annular band using a laser ablation technique. In some cases, the laser lathing and/or the laser ablation can form one or more micro-bumps along the inner surface of the annular band. In some cases, the annular band can undergo laser lathing and/or the laser ablation simultaneously. The one or more grooves, blind grooves, holes, blind holes, and/or micro-bumps along the inner surface of the annular band can beneficially allow for aspiration to be applied in one or more modes as described herein.

Any of the annular bands described herein can include or more bumps, also referred herein to as islands. The one or more bumps can be positioned along the inner surface of the annular band. The bumps can extend radially inward from the inner surface of the annular band so that at least a portion of the bumps is exposed to the catheter lumen. The bumps can help maintain interventional devices (such as catheters and/or wires) being advanced through the catheter having the annular band centered within the catheter. This can prevent the interventional devices from clogging and/or blocking the interior surface of the annular thereby allowing the annular band to apply aspiration as described herein.

The inner surface of any of the annular bands described herein can be surface roughened to create a texture along the inner surface of the annular band. For instance, the inner surface of the annular band can be surface roughened to form one or more grooves, blind grooves, holes, blind holes, micro-bumps, and/or a swaging pattern. In some cases, the annular band can include a corrugated pattern.

In some cases, the annual bands described herein can be manufactured using a Swiss screw method. Swiss screw machining can be used to form one or more grooves, blind grooves, holes, blind holes, and/or micro-bumps along the annular band. In some cases, raw stock tubing can be processed using the Swiss screw machining to form layers having different thicknesses. After the raw stock tubing is processed using the Swiss screw machining, the raw stock tubing can undergo a laser drilling process, as previously described herein, to form additional grooves, blind grooves, holes, blind holes, and/or micro-bumps. In some cases, a multilayered drawn filled tube (DFT) tubing can be formed into an annular band. The multilayered DFT tubing can provide areas of radiopacity that can beneficially be used as standoffs.

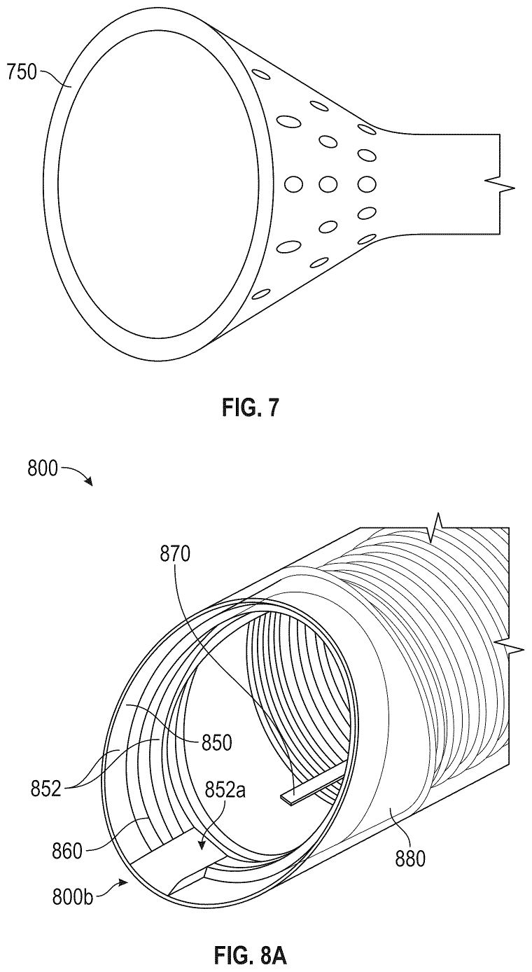

In some cases, the annular band 750 can include a funnel shape, as shown in FIG. 7. In some cases, the annular band 750 can include a funnel shape. The annular band 750 can beneficially prevent ingested clots from backing out after initial engagement. The annular band 750 can include a tooth exposed to the lumen of the catheter. The tooth can engage aspirated clot and prevent the aspirated clot from backing out into the vasculature after initial engagement. The funnel shape can beneficially improve clot ingestion.

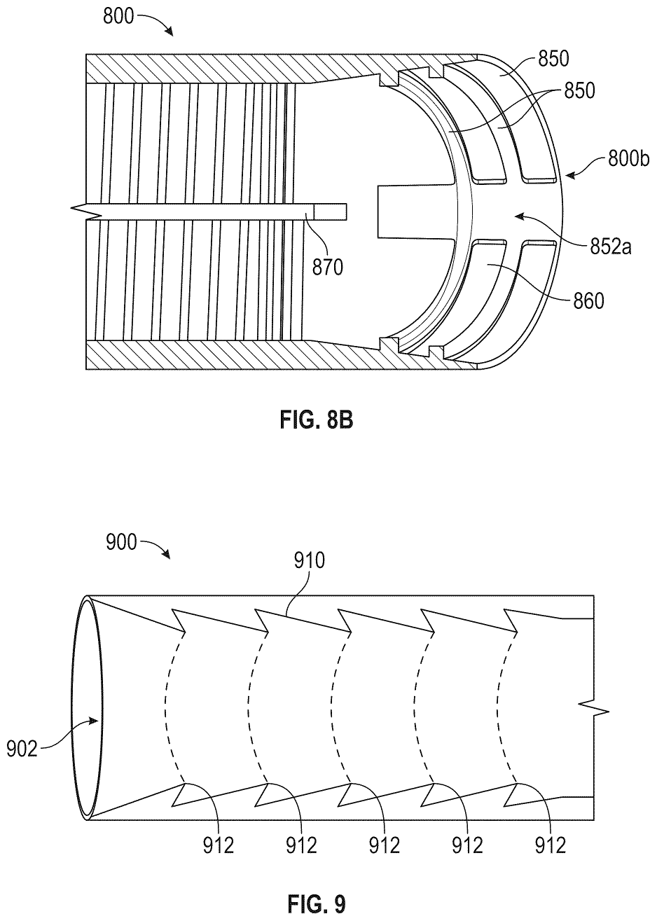

FIGS. 8A and 8B show another example of a catheter 800 with a molded tip 850. In some cases, the molded tip 850 can be formed from a polymer material. The molded tip 850 can include one or more ridges or rings 852. The rings 852 can be spaced apart from each other. In some embodiments, the molded tip 850 can include one or more grooves 860. For example, a groove 860 can separate each pair of rings 852. In some cases, each ring 852 can include a gap 852a. The groove 860 and/or the gap 852a can allow aspiration flow within the catheter to flow both longitudinally along the lumen of the catheter 800 as well as radially outwardly (e.g., when a clot is engaged/stuck on the distal end of the catheter 800). In some cases, the gap 852a can prevent the molded tip 850 from folding when, for example, the catheter 800 is being advanced and/or retracted, and/or when aspiration is being applied. This can beneficially prevent blocking of the central lumen of the catheter 800 during a procedure. An inner diameter of the molded tip 850 can decrease in a proximal direction such that the inner diameter of the molded tip 850 is larger at a distal end 800b of the catheter 800. The tapering inner diameter of the molded tip 850 can result in the molded tip 850 having a funnel-shaped interior surface. In some cases, the catheter 800 can have a beveled distal face. The one or more rings 852 can be angled to match the angle of the bevel distal face. The one or more rings 852 can provide structural support to the molded tip 850 to maintain the profile of the molded tip 850 (e.g., during aspiration or as the catheter is traversed through tortuous or narrowed vasculature). The rings can 852 can prevent a clot from disengaging from the tip 850.

The catheter 800 can include a reinforcement element 870. The reinforcement element 870 can include one or more axially extending mono strands and/or multi strand filaments. In some cases, the reinforcement element 870 can include one or more wires. The reinforcement element 870 may be axially placed inside the catheter wall near the distal end of the catheter 800. The reinforcement element 870 can be secured to the catheter 800 on one end and to the molded tip 850 on the other end. The reinforcement element 870 can be secured to a long edge of the molded tip 850. In some cases, the reinforcement element 870 can prevent detachment of the molded tip 850 from the catheter 800 when, for example, the catheter 800 is retracted and/or pulled (e.g., when the catheter is being proximally retracted through tortuous or narrowed vasculature).

The molded tip 850 can include a marker band 880. The marker band 880 can include a platinum material. The marker band 880 can be radiopaque, which can beneficially allow visualization of the catheter 800 and/or the molded tip 850 under fluoroscopy.

An inner most diameter of the molded tip 850 may be substantially similar to or at least as large as an inner diameter of the catheter proximal to the molded tip 850, for example, so that it does not affect compatibility with other devices that may be traversed through the catheter.

FIG. 9 shows another example of a catheter 900. The catheter 900 can include an inner liner 910. The inner liner 910 can include a plurality of wrinkles or teeth 912. The plurality of teeth 912 can be exposed to a lumen 902 of the catheter 900. The teeth 912 may allow clots aspirated by the catheter 900 to advance in a proximal direction (e.g., towards the source of aspiration and away from the patient) but prevent clots from returning to the vasculature of the patient (e.g., due to the orientation of the teeth). For example, as clots advance through the lumen 902 of the catheter 900, the teeth 912 can prevent accidental return of the clot into the vasculature of a patient by engaging and locking the clot. The teeth 912 may be angled in proximal direction (e.g., to a proximal pointed end) so that a clot moving proximally can slide along a generally smooth surface, but a clot moving in the distal direction would catch on steep angled portions (e.g. the proximal pointed ends) of the teeth 912. The plurality of teeth 912 can extend along a longitudinal axis of the inner liner 910. In some cases, the plurality of teeth 912 can form a continuous and/or interrupted spiral pattern along an inner surface of the inner liner 910. The one or more teeth 912 can beneficially allow the catheter 900 to apply aspiration similar to how the annular bands described herein apply aspiration (e.g., in one or more modes). Although described as part of a liner 910, in some embodiments, the teeth 912 may be formed in a wall of the catheter 900.

In some embodiments, a portion of a catheter, liner, and/or insert may have a profile shaped, dimensioned, or otherwise configured to increase a surface area contacting a clot (e.g., in comparison to a cylindrical profile). For example, any of the catheters described herein can include a barb exposed to the main lumen of the catheter. The barb can beneficially engage aspirated clots thereby preventing the aspirated clots from backing out into the vasculature upon aspiration and/or engagement of the same.

Any of the catheters disclosed herein, including those having an annular band and/or a porous tip, can include a stent-like backbone or support as described further herein (also referred herein to as a stent). In some cases, the stent or backbone can be made from a metallic material. The stent or backbone can be positioned along the body of the catheter and can provide support to the catheter. A catheter with a stent as described herein may not require a hypotube. In some cases, the stent-like backbone can be integral with the annular band and/or porous tip, if provided. For instance, a distal end of the stent-like backbone can include the annular band and/or the porous tip. Having the annular band and/or porous tip be integral with the stent-like backbone can prevent the annular band and/or porous tip from disengaging from the catheter. This is especially beneficial when, for example, the catheter is being retracted and/or pulled. The stent-like backbone can also provide support to the catheter when the catheter is advanced through the vasculature. The stent-like backbone can prevent the round-shape of a catheter from transforming into an oval shape when, for example, the catheter is exposed to constant pushing, pulling, and/or bending.

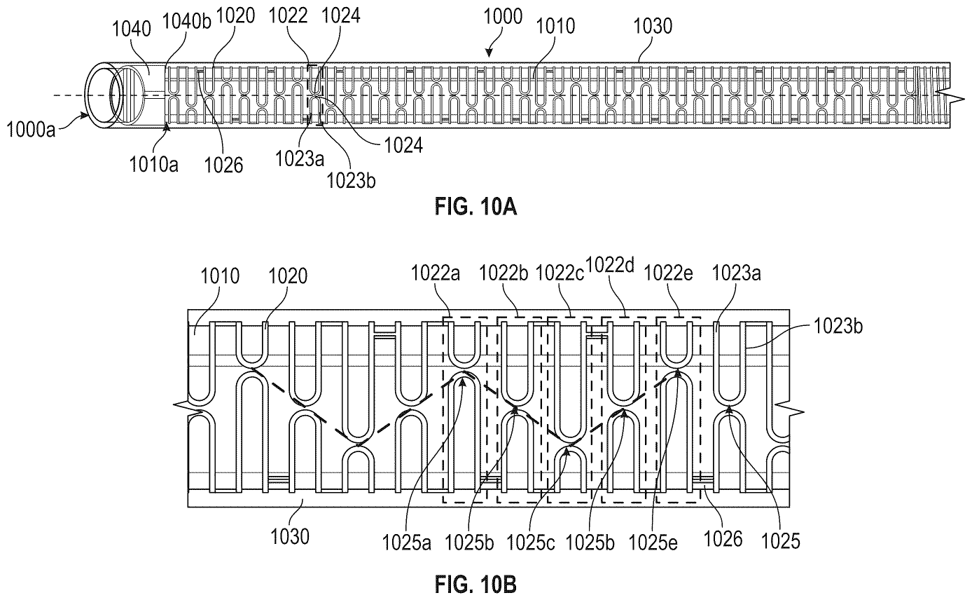

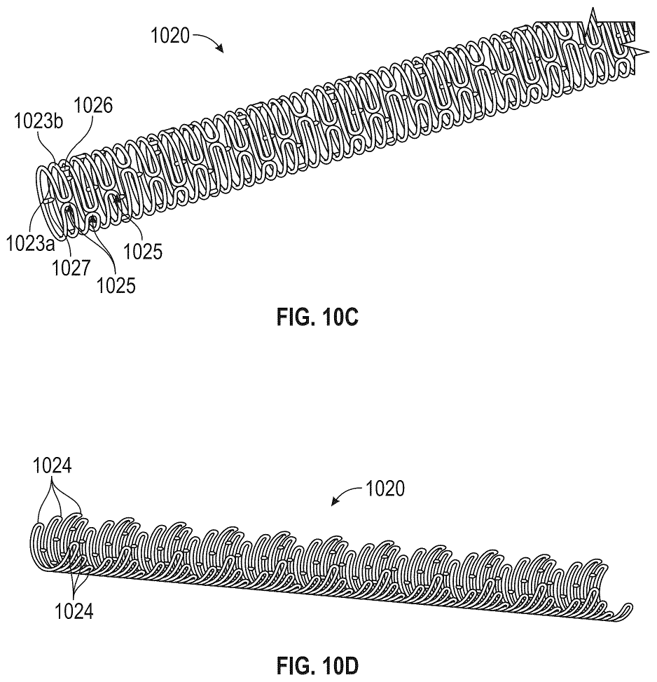

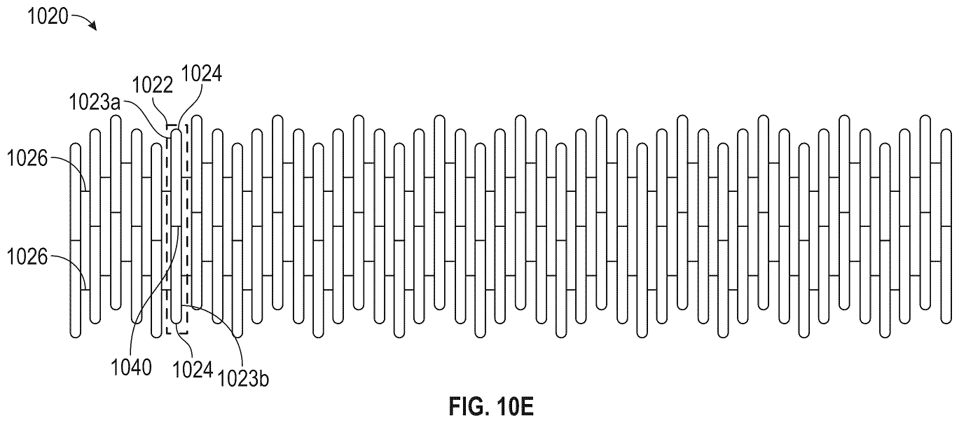

FIGS. 10A and 10B show an example of a catheter having a stent-like backbone. The catheter 1000 can include an inner liner 1010, a stent or backbone 1020, and/or an outer layer 1030. In some cases, the backbone 1020 can be positioned between the inner liner 1010 and the outer layer 1030. The outer layer 1030 can include a jacket. The jacket can include one or more polymer segments having the same or different properties. For example, the segments can be made from different materials. The use of different materials can allow the flexibility of the catheter 1000 to vary along the length of the catheter 1000. For example, it may be beneficial for distal segments of the catheter 1000 to be more flexible than proximal segments of the catheter 1000.

The catheter 1000 can include a marker band 1040. In some cases, the marker band 1040 can be positioned proximal to a distal end 1000a of the catheter 1000. The inner liner 1010 can extend from a proximal end of the catheter 1000 to a distal end of the catheter 1000. In some cases, the inner liner 1010 can extend from a proximal end of the catheter 1000 to a proximal edge 1040b of the marker band 1040, such that a distal end 1010a of the inner liner 1010 is proximal to the proximal edge 1040b of the marker band 1040. In some cases, the inner liner 1010 can extend from a proximal end of the catheter 1000 past the marker band 1040 to terminate at or proximal to the distal end 1000a of the catheter 1000.

As further described below, the backbone 1020 can include a c-shape. For instance, the backbone 1020 can include a metallic support having a continuous gap extending along a length of the backbone 1020 to define a c-shaped structure (e.g., in a transverse cross-section view). The continuous gap can be linear (e.g., where the gap is parallel to a longitudinal axis of the backbone 1020) and/or non-linear.

As shown in FIG. 10B, the backbone 1020 can include a plurality of open rings 1022. Each open ring 1022 can include a first band 1023a and a second band 1023b. The first band 1023a and the second band 1023b can be connected via a beam 1024 and/or a connection 1027. In some cases, the first band 1023a and the second band can be connected via two beams 1024 and a connection 1027. The connection 1027 can be oriented parallel to a longitudinal axis of the backbone 1020. In some cases, the connections 1027 are not parallel to the longitudinal axis of the backbone 1020. This arrangement can improve the compressibility and/or flexibility of the backbone 1020. This can allow the catheter 1000 to better accommodate bending and/or movement without compromising the structural integrity of the catheter 1000.

The beams 1024 can include an arc-shape connecting the ends of the first band 1023a and the second band 1023b. The beams 1024 can be separated by a gap 1025. As further described below, the gaps 1025 can beneficially allow the backbone 1020 to be opened for loading. In some cases, the gap 1025 and the connection 1027 of each open ring 1022 can be spaced about 180° apart from each other around the longitudinal axis of the backbone 1020. Each open ring 1022 can be connected to at least another open ring 1022 via one or more struts 1026. In some cases, each open ring 1022 is connected to another open ring 1022 via a pair of struts 1026, which can be spaced about 180° apart from each other around the longitudinal axis of the backbone 1020. For example, each pair of struts 1026 can be spaced apart from each other around the longitudinal axis of the backbone 1020 by about 45°, 60°, 90°, 120°, 150°, and/or 180°. In some cases, each pair of struts 1026 can be spaced apart from each other around the longitudinal axis of the backbone 1020 by between about 45° and about 180°, between about 60° and about 165°, between about 75° and about 150°, between about 90° and about 135°, and/or between about 105° and about 120°. Each strut 1026 can be parallel to the longitudinal axis of the backbone 1020. In some cases, the struts 1026 are not parallel to the longitudinal axis of the backbone 1020.

The backbone 1020 can be designed to achieve a specific flexibility profile for the catheter 1000. The arrangement and dimensions of the open rings 1022, the beams 1024, the connections 1027, and/or the struts 1026 can be tailored to provide varying degrees of stiffness along the length of the catheter 1000. The backbone 1020 can be modified based on clinical needs to achieve the desired stiffness and flexibility profile.

Adjacent pairs of gaps 1025 can be offset from each other from about15° to about 65° (e.g., by about 25°, 35°, 450, and/or 55°). For example, a first open ring 1022a can include a first gap 1025a at, for example, 35°, as shown in FIG. 10B. A second open ring 1022b can include a second gap 1025b at 0°, so that the first gap 1025a and the second gap 1025b are offset by about 35°. A third open ring 1022c can include a third gap 1025cat −35°, so that the third gap 1025c and the second gap 1025b are offset by about 35°. A fourth open ring 1022d can include a fourth gap 1025d at 0°, so that the fourth gap 1025d and the third gap 1025c are offset by about 35°. A fifth open ring 1022e can include a fifth gap 1025e at 35°, so that the fifth gap 1025e and the fourth gap 1025d are offset by about 35°. The pattern created by the first, seconds, third, fourth gaps, can repeat over the length of the backbone 1020 defining a W pattern along the length of the backbone 1020, as shown in FIG. 10B. In some cases, adjacent pairs of gaps 1025 can be offset from each other from about 15° to about 75°. (e.g., 15°, 20°, 25°, 30°, 45°, 60°, 70°). Offset gaps 1025 can collectively form a non-linear continuous gap along the backbone 1020. In alternative embodiments, the gaps 1025 may not be offset and may form a linear continuous gap along the backbone 1020.

The non-linear continuous gap extending along the length of the backbone 1020 can prevent material bunching when the catheter 1000 is subjected to bending. By offsetting the gaps in a repeating W pattern along the length of the backbone 1020, the backbone 1020 can distributes flexural stresses more evenly and/or avoid the formation of a preferential bending plane. This can allow the catheter 1000 to maintain a consistent flexibility and/or shape retention in all directions, thereby enhancing navigability through tortuous vasculature and/or reducing the risk of kinking or deformation during a procedure.

FIGS. 10C-10E show isolated views of the backbone 1020. The backbone 1020 can be opened for loading onto a catheter structure. For example, the backbone 1020 can be secured to the inner liner 1010 by opening the backbone 1020 along the gaps 1025, as shown in FIG. 10D, positioning the backbone 1020 over the inner liner 1010, and closing the backbone 1020. With the backbone 1020 positioned over the inner liner 1010, the outer layer 1030 can be positioned over the inner liner 1010 and the backbone 1020. Catheters with a hypotube construction typically require an inner diameter of the hypotube to be larger than an outer diameter of the inner liner. The loading gap between the inner liner and the hypotube results in a catheter with a larger outer diameter. Since the backbone 1020 can be secured to the inner liner 1010, and does not need to be slid onto the inner liner 1010, ta loading gap is not required. This can beneficially reduce the overall outer diameter of the catheter 1000. The interference fit between the backbone 1020 and the inner liner 1010 can enhance the hoop strength of the catheter 1000, which can provide improved resistance to kinking. The interference fit can also help maintain proper alignment during assembly, ensuring that the components remain securely in place and reducing the risk of misalignment or shifting throughout the manufacturing process and clinical use.

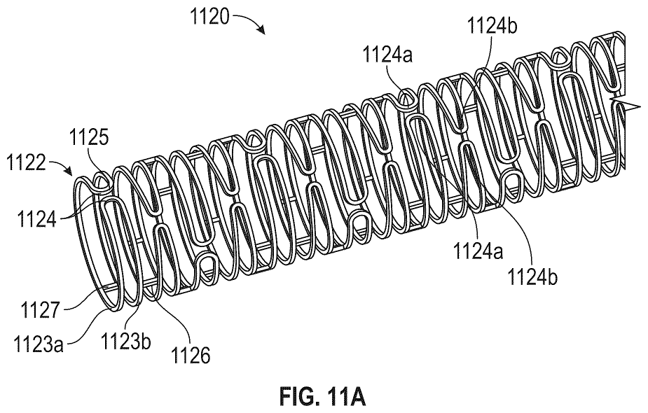

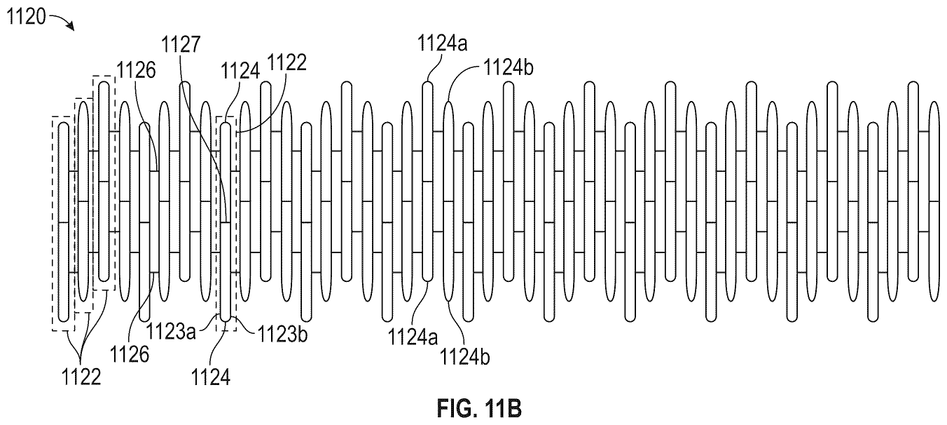

FIGS. 11A and 11B show another example of a backbone or stent for a catheter. Except as otherwise described herein, the backbone 1120 can generally include any of the same and/or similar features and functions as any of the other backbones described herein (e.g., the backbone 1020). For instance, the backbone 1120 can be positioned between an inner liner and an outer layer of a catheter (for example catheter 1000). In some cases, and as further described below, the beams of the backbone 1120 can have different shapes.

The backbone 1120 can include a plurality of open rings 1122. Each open ring 1122 can include a first band 1123a and a second band 1123b. The first band 1123a and the second band 1123b can be connected via a beam 1124 and/or a connection 1127. In some cases, the first band 1123a and the second band can be connected via two beams 1124 and a connection 1127. The connection 1127 can be oriented perpendicular to a longitudinal axis of the backbone 1120. The beams 1124 can be separated by a gap 1125. The gaps 1125 can beneficially allow the backbone 1120 to be opened for loading. In some cases, the gap 1125 and the connection 1127 of each open ring 1122 can be spaced about 180° apart from each other around the longitudinal axis of the backbone 1120. Each open ring 1122 can be connected to at least another open ring 1122 via one or more struts 1126. In some cases, each open ring 1122 is connected to another open ring 1122 via a pair of struts 1126, which can be spaced about 180° apart from each other around the longitudinal axis of the backbone 1120. For example, the each pair of struts 1126 can be spaced apart from each other around the longitudinal axis of the backbone 1020 by about 45°, 60°, 90°, 120°, 150°, and/or 180°. Each strut 1126 can be oriented perpendicular to a longitudinal axis of the backbone 1120.

Unlike the beams 1024 of the backbone 1020, the shape of the beams 1124 can change along the length of the backbone 1120. For example, the backbone 1120 can include a pattern in which a first set of beams 1124a has a first shape and a second set of beams 1124b includes a second shape. The second set of beams 1124b can be directly adjacent to the first set of beams 1124a. In some cases, the first shape can be different than the second shape. For instance, the first shape of the first set of beams 1124a can include a wide arc-shape, and the second shape of the second set of beams 1124b can include a narrow arc-shape. The reduced width of the narrow arc-shaped beams 1124 can reduce congestion when, for example, the stent is bent. The smaller radius of the narrow arc-shaped beams 1124 can improve bending by allowing the backbone 1110, and the catheter that the backbone 1124 is secured to, to move more freely. Because the apex of the narrow arc-shaped beams 1124 is smaller, it can occupy less space and be less likely to interfere with or bump into neighboring arcs when the backbone 1124 and/or the catheter bends. In some cases, the backbone 1120 can include an alternating pattern in which every other set of beams 1124 includes a wide-arc shape and/or a narrow-arc shape, as shown in FIGS. 11A and 11B. In some cases, however, the entire backbone 1120 can include a set of beams having a narrow-arc shape.

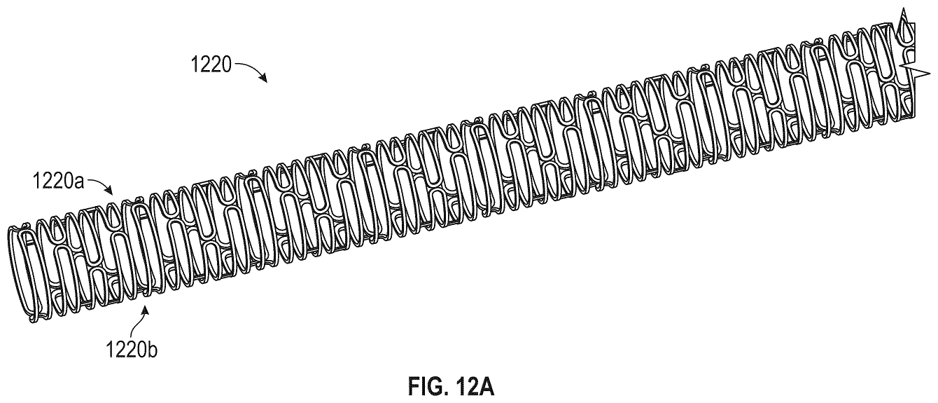

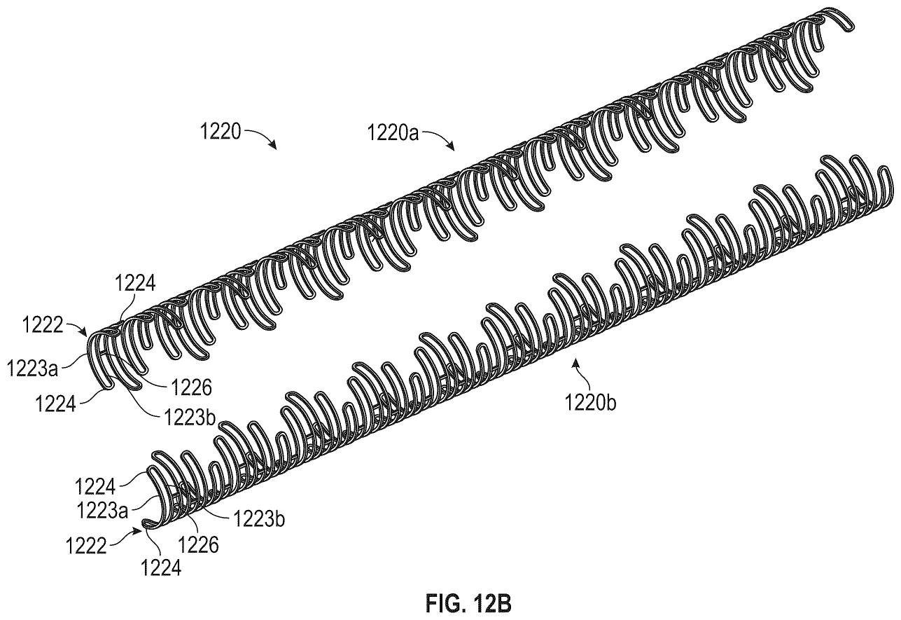

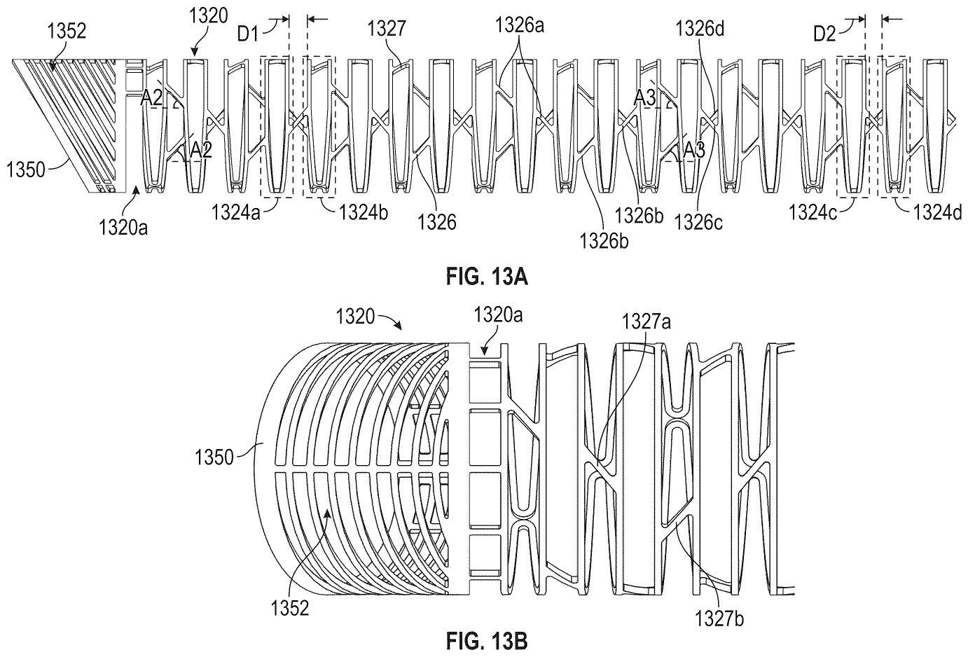

FIGS. 12A and 12B show another example of a backbone or stent 1220 for a catheter. Except as otherwise described herein, the backbone 1220 can generally include any of the same and/or similar features and functions as any of the other backbones described herein. Unlike the backbone 1020 and the backbone 1120, which are described in relation to FIGS. 10A-10E and FIGS. 11A-11B respectively, the backbone 1220 can include two discrete portions. For instance, the backbone 1120 can include a first portion 1220a and a second portion 1220b. Having two discrete portions can beneficially allow the backbone 1220 to be easily secured to an inner liner and/or catheter without the need to slide the backbone 1220 over the inner liner during construction of the catheter.

Each of the first portion 1220a and the second portion 1220b can include a plurality of ringlets 1222. The ringlets 1222 can be similar to the open rings 1022 and/or the open rings 1122. Each ringlet 1222 can include a first band 1223a and a second band 1223b. The first band 1223a and the second band 1223b can be connected via a beam 1224. In some cases, the first band 1223a and the second band 1223b can be connected via two beams 1224. The beams 1224 can include an arc-shape connecting the ends of the first band 1223a and the second band 1223b. Each ringlet 1222 can be connected to at least another ringlet 1222 via one or more struts 1226. In some cases, each ringlet 1222 can be connected to another ringlet 1222 via a single strut 1226. Each strut 1226 can be oriented perpendicular to a longitudinal axis of the backbone 1020.

The backbone 1220 can be secured to a catheter by mounting the first portion 1220a and the second portion 1220b over the catheter and tightening the first portion 1220a and the second portion 1220b to the catheter. In some cases, the backbone 1220 can be secured to an inner liner of the catheter. The first portion 1220a and the second portion 1220b of the backbone 1220 can be positioned over the catheter so that there is a gap 1225 between the beams 1224 of a ringlet 1222 on the first portion 1220a and the beams 1224 of a ringlet 1222 on the second portion 1220b.

Any of the backbones described herein, including the backbones 1020, 1120, and/or 1220, can be integrated into a catheter having an annular and/or a porous tip. In such cases, the annular band, such as the annular band 150, the annular band 250, the annular band 350, the annular band 350′, and/or the annular band 550, can be secured to the backbone. For example, the annular band can be secured to a distal end of the backbone. This can beneficially prevent the annular band from detaching from the catheter when, for example, the catheter is advanced, retracted, and/or bent along the vasculature.

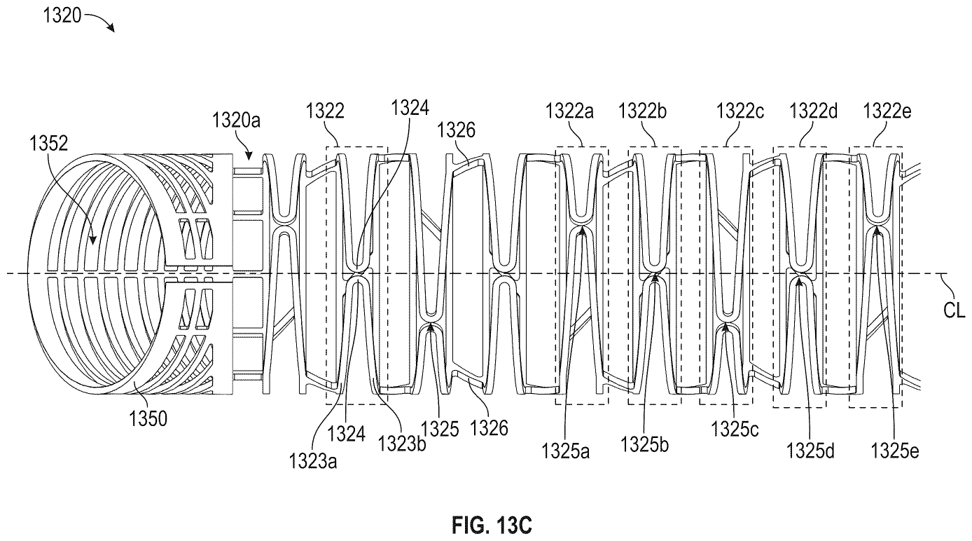

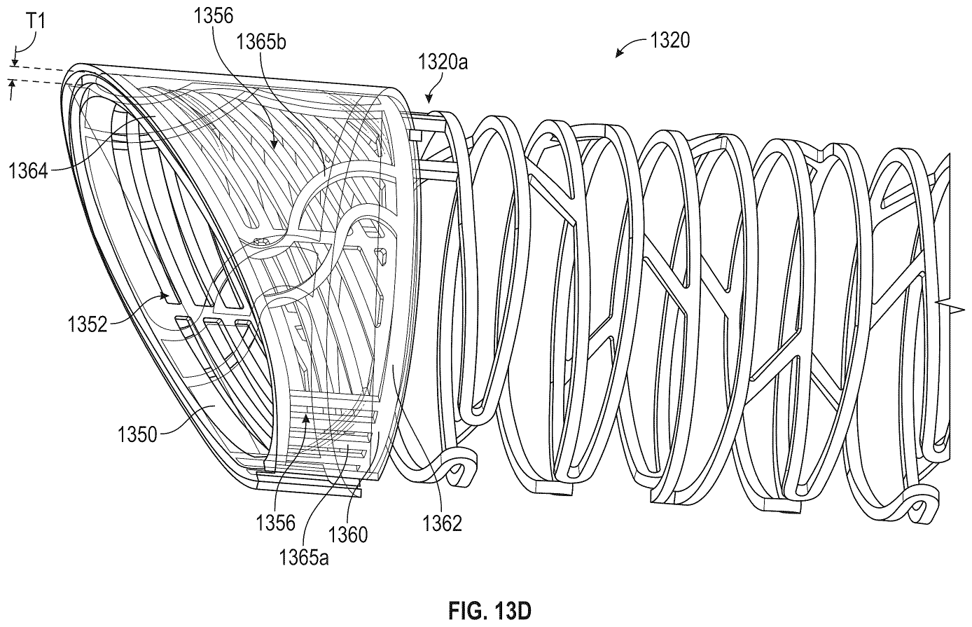

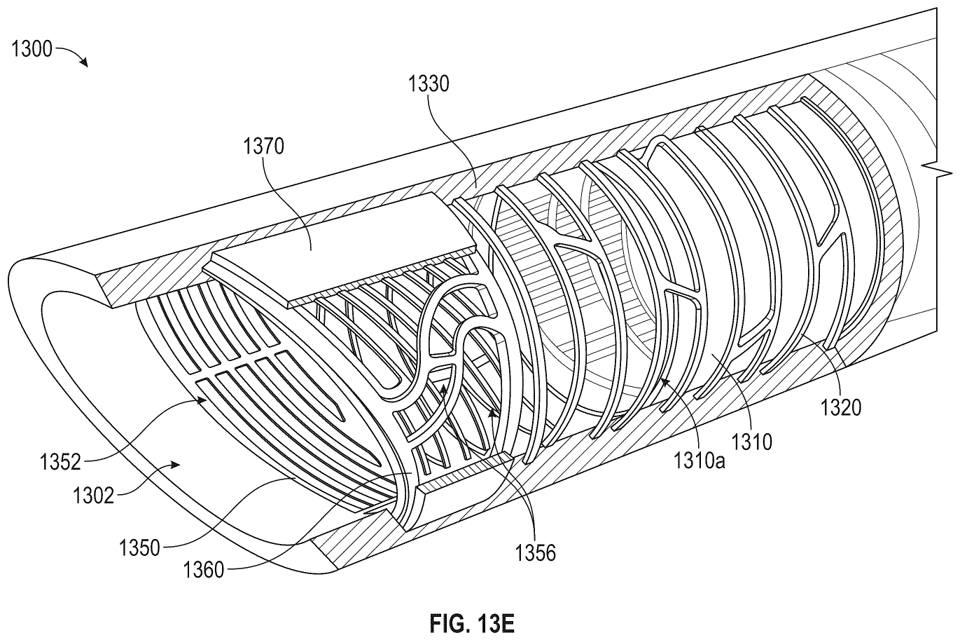

FIGS. 13A-13C show another example of a backbone for a catheter. The backbone shown in FIGS. 13A-13C can be used with any of the catheters described herein. In some cases, a distal end 1320a of the backbone 1320 can be secured to an annular band 1350. The annular band 1350 can be similar or identical to the any of the annular bands described herein, and can include one or more slots 1352. In some cases, the backbone 1320 can include a c-shape. For instance, the backbone 1320 can include a metallic support having a continuous gap extending along a length of the backbone 1320 to define a c-shaped structure (e.g., in a transverse cross-section view). The continuous gap can be linear (e.g., where the gap is parallel to a longitudinal axis of the backbone 1320) and/or non-linear.

As shown in FIG. 13C, the backbone 1320 can include a plurality of open rings 1322. Each open ring 1322 can include a first band 1323a and a second band 1323b. The first band 1323a and the second band 1323b can be connected via a beam 1324 and/or a connection 1327. In some cases, the first band 1323a and the second band 1323b can be connected via two beams 1324 and a connection 1327. In some cases, the orientation of the connection 1327 is not parallel to (e.g., at a non-parallel angle relative to) the longitudinal axis of the backbone 1320. The orientation of the connections 1327 can provide axial and torsional compliance. The non-parallel connections 1327 can allow adjacent rings to advance, retract, and/or rotate relative to each other when the backbone is 1320 is bent. This arrangement can improve the compressibility and/or flexibility of the backbone 1320. This can allow the catheter which the backbone 1320 is a part of to better accommodate bending and/or movement without compromising the structural integrity of the catheter.

In some cases, the connections 1327 can be arranged in an alternating, non-parallel pattern along the backbone 1320. For example, as shown in FIG. 13B, a first pair of connections 1327a can be oriented downward (in a distal to proximal direction). A second pair of connections 1327b, which can be positioned next to the first pair of connections 1327a, can be oriented upward (in a distal to proximal direction). The pattern defined by the first pair of connections 1327a and the second pair of connections 1327b can repeat over the length of the backbone 1320. This pattern can create a wave-like sequence around the circumference of the backbone 1320 that can promote balanced circumferential flexure, add compressibility, and/or disrupt single preferential bending planes. By alternating the orientation of successive connection pairs, the backbone 1320 can distribute bending stresses more uniformly, reduce jacket or liner bunching during, and/or maintain consistent trackability through tortuous vasculature.

The angle of the connections 1327 can vary along the length of the backbone 1320 to tune flexibility and compressibility along the length of the backbone 1320. For example, the angle of the connections 1327 along a distal portion of the backbone 1320 can be larger than the angle of the connections 1327 along a proximal portion of the backbone. The angle of the connections 1327 can be measured relative to a line parallel to the longitudinal axis of the backbone 1320. An angle of the connections 1327 along the distal portion of the backbone 1320 can range from about 40° to about 70° (for example, from about 50° to about 60°). An angle of the connections 1327 along the proximal portion of the backbone 1320 can range from about 20° to about 50° (for example, from about 30° to about 40°). The larger angle of the connections 1327 along the distal portion of the backbone 1320 can increase compressibility and/or promote easier bending and trackability in tortuous distal anatomy. The smaller angle of the connections 1327 along the proximal portion of the backbone 1320 can provide greater axial stiffness and/or support, which can improve pushability and/or shape retention closer to the operator.

The beams 1324 can include an arc-shape connecting the ends of the first band 1323a and the second band 1323b. The beams 1324 can be separated by a gap 1325. As further described below, the gaps 1225 can beneficially allow the backbone 1220 to be opened for loading. In some cases, the gap 1325 and the connection 1327 of each open ring 1322 can be spaced between about 150° to about 180° apart from each other around the longitudinal axis of the backbone 1320. For example, the gap 1325 and the connection 1327 of each open ring 1322 can be spaced about 180° apart from each other around the longitudinal axis of the backbone 1320.

Each open ring 1322 can be connected to at least another open ring 1322 via one or more struts 1326. In some cases, each open ring 1322 is connected to another open ring 1322 via a pair of struts 1326, which can be spaced between about 150° to about 180° apart from each other around the longitudinal axis of the backbone 1320. For example, each pair of struts 1326 can be spaced apart from each other around the longitudinal axis of the backbone 1320 by about 45°, 60°, 90°, 120°, 150°, and/or 180°. In some cases, each pair of struts 1326 can be spaced apart from each other around the longitudinal axis of the backbone 1320 by between about 45° and about 180°, between about 60° and about 165°, between about 75° and about 150°, between about 90° and about 135°, and/or between about 105° and about 120°. In some cases, an orientation of each strut 1326 is not parallel to (e.g., at a non-parallel angle relative to) the longitudinal axis of the backbone 1320.

In some cases, the struts 1326 can be arranged in an alternating, non-parallel pattern along the backbone 1320. For example, as shown in FIG. 13A, a first pair of consecutive struts 1326a on a first side of the backbone 1320 can be oriented downward (in a distal to proximal direction). A second pair of consecutive struts 1326b on the first side of the backbone 1320, which can be positioned next to the first pair of consecutive struts 1326a, can be oriented upward (in a distal to proximal direction). The pattern defined by the first pair of consecutive struts 1326a and the second pair of consecutive struts 1326b can repeat over the length of the backbone 1320. This pattern can create a wave-like sequence around the circumference of the backbone 1320 that can promote balanced circumferential flexure, add compressibility, and/or disrupt single preferential bending planes. By alternating the orientation of pairs of consecutive struts, the backbone 1320 can distribute bending stresses more uniformly, reduce jacket or liner bunching during, and/or maintain consistent trackability through tortuous vasculature. In some embodiments, each consecutive strut may alternate in direction. In other embodiments, the struts can alternate in consecutive sets of three or more struts. Each strut 1326 positioned of the second side of the backbone 1320, can be oriented in an opposite direction relative to a corresponding strut 1326 on the first side of the backbone 1320. For example, as shown in FIG. 13A, a first strut 1326c positioned on the first side of the backbone 1320 can be oriented upward (in a distal to proximal direction) while a corresponding second strut 1326d on the second side of the backbone 1320 is oriented downward (in a distal to proximal direction).

The angle of the struts 1326 can vary along the length of the backbone 1320 to tune flexibility and compressibility along the length of the backbone 1320. For example, the angle of the struts 1326 along a distal portion of the backbone 1320 can be larger than the angle of the struts 1326 along a proximal portion of the backbone. The angle of the struts 1326 can be measured relative to a line parallel to the longitudinal axis of the backbone 1320. An angle A2 of the struts 1326 along the distal portion of the backbone 1320 can range from about 40° to about 70° (for example, from about 50° to about 60°). The angle of the struts 1326 can be measured relative to a line parallel to the longitudinal axis of the backbone 1320. An angle A3 of the struts 1326 along the proximal portion of the backbone 1320 can range from about 20° to about 50° (for example, from about 30° to about 40°). The larger angle of the struts 1326 along the distal portion of the backbone 1320 can increase compressibility and/or promote easier bending and trackability in tortuous distal anatomy. The smaller angle of the struts 1326 along the proximal portion of the backbone 1320 can provide greater axial stiffness and/or support, which can improve pushability and shape retention closer to the operator.

In some cases, the spacing between adjacent bands of consecutive open rings can decrease in a proximal direction. For example, as shown in FIG. 13A, a distance D1 between a first open ring 1324a and a second open ring 1324b, which are positioned on a distal portion of the backbone 1320, can be larger than a distance D2 between a third open ring 1324c and a fourth open ring 1324d, which are positioned on a proximal portion of the backbone 1320. The larger spacing between adjacent open rings toward the distal portion of the backbone 1320 can enhance compressibility. The smaller spacing between adjacent open rings toward the proximal portion of the backbone 1320 can provide greater axial support and/or improve pushability.

The backbone 1320 can be designed to achieve a specific flexibility profile for a catheter. The arrangement and dimensions of the open rings 1322, the beams 1324, the connections 1327, and/or the struts 1326 can be tailored to provide varying degrees of stiffness along the length of a catheter. The backbone 1320 can be modified based on clinical needs to achieve the desired stiffness and flexibility profile.

Adjacent pairs of gaps 1325 can be offset from each other from about 15° to about 65° (e.g., by about 25°, 35°, 45°, and/or 55°). For example, a first open ring 1322a can include a first gap 1325a positioned between about 35° to about 45° from a center line CL, as shown in FIG. 13C. A second open ring 1322b can include a second gap 1325b at about 0° relative to the center line CL (e.g., along the center line CL) so that the first gap 1325a and the second gap 1325b are offset by between about 35° and about 45°. A third open ring 1322c can include a third gap 1325c positioned between about-35° to about-45° from the center line CL, so that the third gap 1325c and the second gap 1325b are offset by between about 35° to about 45°. A fourth open ring 1322d can include a fourth gap 1325d at about 0° relative to the center line CL (e.g., along the center line CL) so that the fourth gap 1325d and the third gap 1325c are offset by between about 35° and about 45°. A fifth open ring 1322e can include a fifth gap 1325e positioned between about 35° to about 45° from the center line CL, so that the fifth gap 1325e and the fourth gap 1325d are offset by offset by between about 35° to about 45°. The pattern created by the first, second, third, fourth, and fifth gaps, can repeat over the length of the backbone 1320 defining a “W” pattern along the length of the backbone 1320, as shown in FIG. 13C. In some cases, adjacent pairs of gaps 1325 can be offset from each other between about 15° to about 75°. (e.g., 15°, 20°, 25°, 30°, 45°, 60°, 70°). Offset gaps 1325 can collectively form a non-linear continuous gap along the backbone 1320. In alternative embodiments, the gaps 1325 may not be offset and may form a linear continuous gap along the backbone 1320.

The non-linear continuous gap extending along the length of the backbone 1320 can prevent material bunching when a catheter is subjected to bending. By offsetting the gaps in a repeating W pattern along the length of the backbone 1320, the backbone 1320 can distribute flexural stresses more evenly and/or avoid the formation of a preferential bending plane. This can allow a catheter to maintain a consistent flexibility and/or shape retention in all directions, thereby enhancing navigability through tortuous vasculature and/or reducing the risk of kinking or deformation during a procedure.

FIG. 13D shows a partial cross-sectional view of a catheter 1300 that includes an inner liner 1310, a stent-like backbone 1320 positioned over the inner liner 1310, and an outer layer 1330 positioned over the inner liner 1310 and the backbone 1320. The inner liner 1310 can define a lumen 1302 of the catheter 1300. In some cases, a distal end 1310a of the inner liner 1310 can be positioned proximal to the annular band 1350, a spacer 1360, and/or a marker band 1370. The outer layer 1330, which can include one or more polymer segments, can be positioned over the backbone 1320 to form an exterior of the catheter 1300.

The backbone 1320 can be loaded onto a catheter 1300. For example, the backbone 1320 can be secured to an inner liner 1310 by opening the backbone 1320 along the gaps 1325, positioning the backbone 1320 over the inner liner 1310, and closing the backbone 1320. With the backbone 1320 positioned over the inner liner, an outer layer 1330 can be positioned over the inner liner 1310 and the backbone 1320. Catheters with a hypotube construction typically require an inner diameter of the hypotube to be larger than an outer diameter of the inner liner. The loading gap between the inner liner and the hypotube results in a catheter with a larger outer diameter. Since the backbone 1320 can be secured to the inner liner as described herein, and does not need to be slid onto the inner liner, a loading gap is not required. This can beneficially reduce the overall outer diameter of the catheter. The interference fit between the backbone 1320 and the inner liner can enhance the hoop strength of the catheter, which can provide improved resistance to kinking. The interference fit can also help maintain proper alignment during assembly, ensuring that the components remain securely in place and reducing the risk of misalignment or shifting throughout the manufacturing process and clinical use.

FIG. 13E shows a spacer 1360 positioned between the annular band 1350 and a marker band 1370. The spacer 1360, the annular band, and the marker band 1370, can be positioned over the inner liner 1310. The spacer 1360 can define one or more cavities 1356 between the annular band 1350 and the marker band 1370 and/or the outer layer 1330, for radial aspiration and/or clot retention. The spacer 1360 can include an proximal rim 1362 and a distal rim 1364. The proximal rim 1362 and the distal rim 1364 can prevent polymer from the outer layer 1330 from intruding into the one or more cavities 1356 during manufacturing of the catheter 1000. This can beneficially maintain a separation between the annular band 1350 and the marker band 1370 and/or the outer layer 1330.

The proximal rim 1362 and the distal rim 1364 can be connected to each other via one or more support structures. The one or more support structures can include one or more longitudinal dams 1365a that extend generally parallel to a longitudinal axis of the backbone 1320. The one or more longitudinal dams 1365a can prevent polymer flow from flowing into the one or more cavities 1356 during assembly. The one or more support structures can include one or more curved support structures 1365b. The one or more curved support structures 1365b can allow cross-communication between cavities 1356 through the one or more slots 1352 of the annular band 1350. This cross-communication can allow vacuum to be available at each of the one or more cavities 1356 even when one or more of the cavities 1356 is occluded by clot.

In some cases, the spacer 1360 can be formed from a single metallic piece. For example, the metallic piece can be laser cut or etched to define the proximal rim 1362, the distal rim 1364, and/or the one or more support structures. In some cases, a thickness T1 of the spacer 1360 can be between about 0.001 in to about 0.02 in. For example, the thickness of the spacer 1360 can be about 0.001 in, 0.0012 in, 0.0015 in, 0.005 in, 0.01 in, 0.012 in, and/or 0.015 in. Thus, the thickness of the cavities 1356 of the spacer 1360 can be between about 0.001 in to about 0.02 in. For example, the thickness of the cavities 1356 can be about 0.001 in, 0.0012 in, 0.0015 in, 0.005 in, 0.01 in, 0.012 in, and/or 0.015 in.

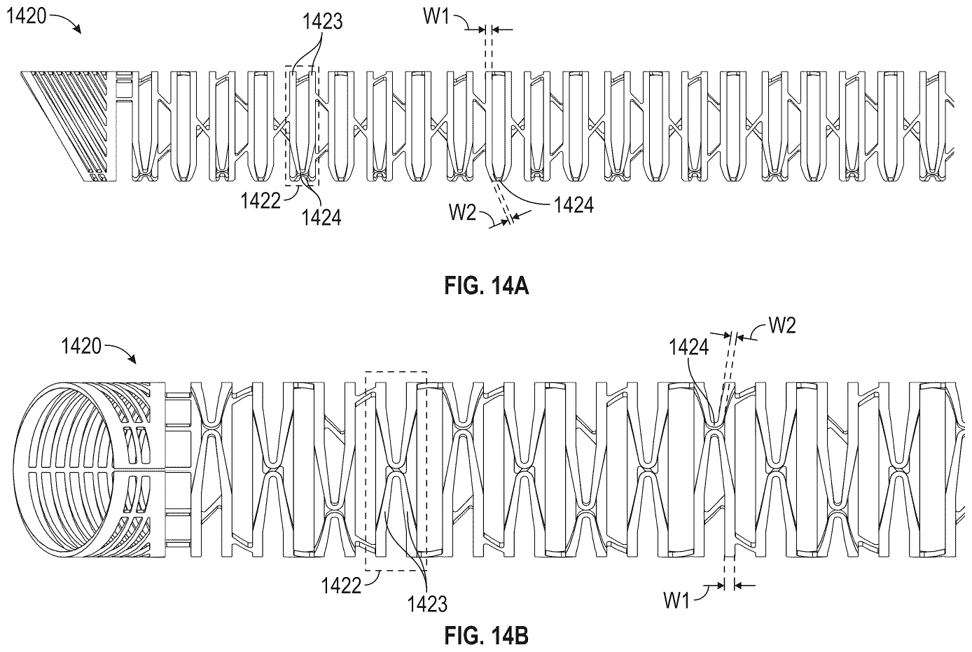

FIGS. 14A-14B show another example of a backbone for a catheter. The backbone 1420 shown in FIGS. 14A-14B can be used with any of the catheters described herein. In some cases, the width of the bands can vary. For example, the width of each band 1423 of the open rings 1422 can taper as the bands 1423 approach the beams 1424. For example, each band 1423 can have a first width W1 ranging from about 0.001 in to about 0.008 in. In some cases, the first width W1 can be about 0.002 in, 0.003 in, 0.004 in, 0.005 in, and/or 0.006 in. A second width W2 of the bands 1423 at portions of the bands 1423 adjacent to the beams 1424 can range from about 0.0005 in to about 0.004 in. In some cases, the second width W2 can be about 0.001 in, 0.002 in, and/or.003 in. In some cases, the first width W1 can be between about 1.1 to about 3 times larger than the second width W2. For example, the first width W1 can be twice as large as that of the second width. The varying width of the bands 1423 can improve radial resistance while maintaining compressibility.

Although the catheters disclosed herein have been described in terms of certain preferred embodiments, they may be incorporated into other embodiments by persons of skill in the art in view of the disclosure herein. For example, other embodiments of aspiration catheters may not include a separate annular band, but may incorporate within an inner wall at the distal end of the catheter a plurality of holes and a gap similar to that of any of the embodiments described herein. The scope of the invention is therefore not intended to be limited by the specific embodiments disclosed herein, but is intended to be defined by the full scope of the following claims.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention. The drawings are for the purpose of illustrating embodiments of the invention only, and not for the purpose of limiting it.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they Can also include any third-party instruction of those actions, either expressly or by implication. For example, actions such as “deploying an instrument sterilized using the systems herein” include “instructing the deployment of an instrument sterilized using the systems herein.” In addition, where features or aspects of the disclosure are described in terms of Markush groups, those skilled in the art will recognize that the disclosure is also thereby described in terms of any individual member or subgroup of members of the Markush group.

The ranges disclosed herein also encompass any and all overlap, sub-ranges, and combinations thereof. Language such as “up to,” “at least,” “greater than,” “less than,” “between,” and the like includes the number recited. Numbers preceded by a term such as “about” or “approximately” include the recited numbers. For example, “about 10 nanometers” includes “10 nanometers.”

Any titles or subheadings used herein are for organization purposes and should not be used to limit the scope of embodiments disclosed herein.

The terms “approximately”, “about”, and “substantially” as used herein represent an amount or characteristic close to the stated amount or characteristic that still performs a desired function or achieves a desired result. For example, the terms “approximately”, “about”, and “substantially” may refer to an amount in certain embodiments that is within less than plus or minus 10% of, within less than plus or minus 5% of, within less than plus or minus 1% of, within less than plus or minus 0.1% of, and within less than plus or minus 0.01% of the stated amount or characteristic.

1. An aspiration catheter comprising:
a tubular body comprising a proximal end, a distal end, and a lumen extending therethrough, wherein the distal end of the tubular body is configured to provide aspiration in a first aspiration mode to apply a tension force to a clot positioned near the distal end along a longitudinal axis of the tubular body;
wherein the aspiration catheter is configured to provide aspiration in a second aspiration mode at least when the clot is positioned within the distal end of the tubular body to apply a second force to the clot along a second axis different from the longitudinal axis.

2. The aspiration catheter of claim 1, wherein the second axis is transverse to the longitudinal axis.

3. The aspiration catheter of claim 1, wherein the second force applied to the clot along the second axis is from within the lumen.

4. The aspiration catheter of claim 1, wherein the lumen provides a first, axial flow path for applying the tension force to the clot, and a structure within the lumen defines a second flow path for applying a radially outwardly directed flow path.

5. The aspiration catheter of claim 4, wherein the structure comprises a barrier within the lumen and spaced radially inwardly apart from an inner surface of the tubular body.

6. The aspiration catheter of claim 5, wherein the barrier is porous.

7. The aspiration catheter of claim 1, further comprising a plurality of holes on an inner surface within the lumen of the tubular body, wherein the plurality of holes are configured to provide aspiration in the second aspiration mode to apply the second force to the clot.

8. The aspiration catheter of claim 7, further comprising an annular band positioned at or near the distal end of the tubular body, the annular band comprising the plurality of holes.

9. The aspiration catheter of claim 1, wherein the first aspiration mode is configured to be active when at least a portion of an opening at the distal end of the tubular body is unobstructed by the clot.

10. The aspiration catheter of claim 9, wherein the second aspiration mode is configured to be active when the clot is corked to the opening at the distal end of the tubular body.

11. The aspiration catheter of claim 10, wherein aspiration provided by the aspiration catheter is configured to transition from the first aspiration mode to the second aspiration mode when the clot gets corked to the opening at the distal end of the tubular body.

12. The aspiration catheter of claim 11, wherein aspiration provided by the aspiration catheter is configured to transition from the second aspiration mode to the first aspiration mode when the clot gets uncorked from the opening at the distal end of the tubular body.

13. The aspiration catheter of claim 1, further comprising a solid surface on an inner surface of the tubular body, wherein the solid surface forms a leak path configured to apply aspiration.

14. A method of aspirating a clot, comprising:
positioning a distal end of an aspiration catheter adjacent a clot;
applying an aspiration force to the clot along a first axis to draw the clot at least partially into the catheter; and
applying a retention force to the clot along a second axis to enhance retention of the clot to the catheter.

15. The method of claim 14, wherein the retention force is applied along the second axis in response to corking of a clot in the catheter.

16. A catheter configured to apply a first, aspiration force and a second, retention force to a clot, comprising:
an elongate, flexible tubular body having a first flow path for applying an axial aspiration force to a clot; and
a second flow path in the tubular body, for applying a radially outwardly directed retention force to the clot.

17. The catheter of claim 16, wherein the second flow path is spaced apart from the first flow path by a porous barrier.

18. The catheter of claim 17, wherein the barrier comprises a mesh.

19. The catheter of claim 17, wherein the barrier comprises a porous wall.

20. The catheter of claim 16, wherein the second flow path is defined between the clot and an inside surface of the flexible tubular body.

21.-78. (canceled)
